(19) United States
(12) Patent Application Publication
(10) Pub. No.: US 2019/0225941 A1
(43) Pub. Date: Jul. 25, 2019

(54) MODULATION OF FUNCTION OF IMMUNE EFFECTOR CELLS

(71) Applicant: OSLO UNIVERSITETSSYKEHUS HF, Oslo (NO)

(72) Inventors: Karl-Johan MALMBERG, Billingstad (NO); Jodie GOODRIDGE, Nesøya (NO)

(21) Appl. No.: 16/336,053

(22) PCT Filed: Sep. 25, 2017
(86) PCT No.: PCT/EP2017/074200

§ 371 Date: Mar. 22, 2019

(30) Foreign Application Priority Data
Sep. 23, 2016 (GB) 1616237.2

(51) Int. Cl. C12N 5/0783; A61K 35/17; C12N 15/113; A61K 31/4709; A61K 31/5377

(52) U.S. Cl. CPC C12N 5/0646; A61K 35/17; C12N 15/113; C12N 2501/999; A61K 31/5377; C12N 2310/14; A61K 31/4709

(57) ABSTRACT

The present invention is directed to methods of modulating the function of granular immune effector cells. It has been discovered that the secretory lysosomes of immune effector cells function as signalling hubs which direct effector functionality of the cells. By increasing or decreasing the signalling potential of the secretory lysosomes of the immune effector cells, effector functionality may be enhanced or reduced, and thus activity of the immune effector cell increased or decreased, respectively. The present invention provides methods for preparing immune effector cells for adoptive cell transfer, in which the cells are contacted with an agent which increases the signalling potential of secretory lysosomes, thus providing enhanced immune effector cells.

The present invention lies in the field of immunology and immunotherapy and is based on the finding that the development of functional potential in cytotoxic and other immune effector cells is linked to the development of secretory lysosomes in the cells, which function as a signalling hub to regulate the effector responses of the cells. Accordingly, the invention is directed to the use of agents which up- or down-regulate the activity of a granular immune effector cell by modulating the size, content and/or number of secretory lysosomes in the said granular immune effector cell, or by otherwise modulating the signalling capacity of the said secretory lysosomes. Such agents may be administered to subjects to achieve this effect, to treat conditions which are responsive to up- or down-regulation of immune effector cell activity and may accordingly have a direct medical use as therapeutic agents, or they may be used ex vivo or in in vitro to up-regulate the activity of immune effector cells for use in adoptive cell transfer therapy, or for research or experimental use (i.e. they may also have a non-medical use). Cells obtained by the in vitro and ex vivo methods of the invention, uses of the cells in therapy and methods of treatment using the cells are also provided.

Eukaryotic cells contain in their cytoplasm acidic secretory lysosomes. In cytotoxic lymphocytes, such as cytotoxic T-cells (CTLs) and natural killer (NK) cells, these secretory lysosomes contain cytotoxins such as perforin, granzymes and granulysin. In the context of cytotoxic lymphocytes, these cytotoxin-containing secretory lysosomes are often referred to as granules. When a cell (e.g. an infected or tumour cell) is targeted for killing by an NK cell or CTL, an immunological synapse is formed between the immune cell and the target cell. Cytolytic secretory lysosomes then polarise (i.e. migrate towards) the synapse, and target cell killing is effected by the process of degranulation, whereby the cytotoxic granules fuse with the cytoplasmic membrane of the cell in which they are contained, releasing their cytotoxins onto the target cell. The cytotoxins enter the target cell and either activate apoptotic pathways or induce cell lysis, thus killing the target cell.

Many leukocytes also release cytokines upon activation, which can play important roles in signalling within the immune system and modulating the immune response. Such cytokines include interferons (IFNs), such as IFNγ, tumour necrosis factors (TNFs), such as TNFα, and interleukins.

NK cells represent an important component of the innate immune system, though they also play a role in adaptive immunity. NK cells provide a rapid immune response to virally-infected cells and respond to tumour formation, primarily by targeting for destruction any cell with reduced expression of the Class I MHC.

The ability of NK cells to sense loss of single MHC class I molecules is based on their stochastic expression of highly polymorphic germ-line-encoded killer cell immunoglobulin-like receptors (KIRs). These receptors are critical for the development of cell-intrinsic functional potential, which enables spontaneous activation upon recognition of target cells with reduced Class I MHC expression. Inhibitory interactions with self-MHCs translate into a predictable quantitative relationship (i.e. a direct correlation) between self-recognition and effector potential, a process termed NK cell education, that is clearly evident in different species and operates through an as yet largely unknown mechanism. Educated NK cells are thus associated with an increased potential for cytotoxic activity, as compared to uneducated or resting NK cells, which are hypo-responsive. Educated NK cells, which have formed inhibitory interactions with self-MHCs and which express self-specific KIRs, may be known as self-specific NK cells. Further discussion of NK cell education can be found in WO 2014/037422, the contents of which is incorporated herein by reference.

NK cell education can be observed at the population level by challenging NK cells with various stimuli, including exposure to target cells lacking MHC expression. A discrete rise in cytosolic Ca²⁺ levels following stimulation of activating receptors can clearly distinguish subsets of self-specific NK cells. Exocytosis of cytolytic granules (and thus cell-killing) is Ca²⁺-dependent, and thus an increase in cytosolic Ca²⁺ levels is associated with activation of NK cell killing functionality. Such an increase in cytosolic Ca²⁺ levels is therefore indicative of educated NK cells. Exocytosis of cytolytic granules by CTLs is also Ca²⁺-dependent, as is cytokine release by CTLs and NK cells.

Speak et al. (Blood 123, 51-60, 2014) showed that in NK cells, release of Ca²⁺ from lysosomes contributes to the calcium release into the cytosol required to drive degranulation.

Davis et al. (Current Biology 22, 2331-2337, 2012) demonstrated that in CTLs, cytosolic Ca²⁺ levels are increased by a combination of inositol 1,4,5-triphosphate (IP₃)-mediated release of Ca²⁺ from the endoplasmic reticulum followed by Ca²⁺ influx into the cell via the STIM/Orai pathway and NAADP-induced release of Ca²⁺ from acidic stores including cytotoxic granules. Rah et al. (Scientific Reports 5, 9482, 2015) demonstrated that NAADP does not induce Ca²⁺ release from lysosomes in NK cells. Instead, they suggest that in NK cells ADP-ribose-induced release of Ca²⁺ from acidic stores, including cytolytic granules, drives both granule polarisation to the immunological synapse and degranulation thereat.

All NK cell effector functions, from adhesion and formation of the immune synapse, induction and secretion of cytokines/chemokines, and exocytosis of cytolytic granules through to in vivo killing of MHC-mismatched targets can be related to the educational status of the NK cell. However, apart from differences in the relative levels and distribution of NK cell receptors at the cell membrane, transcriptional and phenotypic readouts at steady state provide scant differences between responsive, self-specific NK cells and hypo-responsive, non-self-specific NK cells.

In humans, the full cumulative spectrum of NK cell differentiation is revealed through graded increases in the cellular content of both granzyme B and perforin, coupled to increasing capacity for both cytolysis and cytokine production. The basis for progression in functionality within the context of NK cell differentiation is primarily transcriptional and epigenetic, observed in modification of the IFN-γ promoter and in the silencing of signalling adapters such as FCER1γ and Syk in adaptive NK cells that promotes specific signalling pathways, such as CD2, over others. However, the same epigenetic changes do not appear to account for the difference in functional potential between self- and non-self-specific NK cell subsets.

Transfer of mature NK cells from one MHC environment to another results in reshaping of functional potential based on the inhibitory input of the new MHC setting, driven by interactions of the transferred NK cells with host stromal and hematopoietic cells. The short time-scales (hours/days) under which this functional plasticity occurs support the notion that NK cell education can operate independently of differentiation.

The inventors of the present application have discovered that NK cell education is tightly connected to the presence of large, cytotoxin-containing secretory lysosomes within the cell. These secretory lysosomes are also characterised by their high density of membrane components (in particular serglycin) and their close proximity to the centrosome. Accumulation of these secretory lysosomes and the morphological differences in granular structures account for the functional difference between self-specific and non-self-specific NK cell subsets. The inventors of the present application have discovered a novel pathway in NK cells which reduces NK cell functionality (shown in FIG. 18). In the absence of inhibitory KIR signalling, weak agonistic input through activating receptors, including DNAM-1, 2B4, NKG2D and CD16, initiates phosphatidylinositide 3-kinase (PI3K)-dependent phosphorylation of Akt (also known as protein kinase B), thus activating Akt. Akt in turn phosphorylates and activates PIKfyve. PIKfyve, when activated, phosphorylates phosphatidylinositol-3-phosphate to phosphatidylinositol-3,5-bisphosphate, leading to activation of transient receptor potential cation channel mucolipin-1 (TRPML1, also known as Mucolipin-1) and TRPML2. TRPML1 and TRPML2 drive lysosomal fission, leading to loss of NK cell functional potential. Ligation of a ligand to an inhibitory receptor, e.g. a KIR or NKG2A, thus initiating inhibitory signalling from the receptor (as occurs during NK cell education), blocks this signalling cascade at a proximal level through recruitment of protein tyrosine phosphatases, including SHP-1. This results in Vav1 dephosphorylation and thereby shuts down the signalling cascade. In addition, inhibitory receptors induce Crk phosphorylation by the tyrosine kinase c-Abl. Phosphorylated Crk dissociates from cytoskeletal signalling complexes. Shut-down of activating signalling during homeostasis allows the cell to develop large dense core secretory lysosomes with high concentrations of bound Ca²⁺ available as a trigger for a global Ca²⁺-flux in the cell. The capacity of the secretory lysosome to sequester Ca²⁺ in turn leads to its ability to serve as a signalling hub.

The secretory lysosomes of educated NK cells not only contain large amounts of the cytotoxic effector molecules granzyme B and perforin, but also serve as signalling hubs that direct effector responses. These secretory lysosomes were found to propagate surface signalling in NK cells (from cell surface receptors to the endoplasmic reticulum (ER)), ultimately determining the strength of the cells' functional responses, including the ability to secrete cytokine and kill target cells. This signalling function is enabled by the concentration of Ca²⁺ in the secretory lysosomes of educated NK cells, release of which drives signalling cascades which lead to downstream effector responses including degranulation and cytokine production, which are important in the immune response of an NK cell to a target. Thus, Ca²⁺-release from the secretory lysosome initiates a global Ca²⁺ cascade that leads to the downstream effector responses, i.e. it controls, or influences, the activity of the cell.

The inventors of the present application have developed methods by which the above-described discovery may be harnessed, enabling the modulation of NK cell function. Essentially, the methods are based on modulating the ability of the secretory lysosomes to act as a signalling hub (which, in others words, means changing the signalling capacity of the secretory lysosomes). Broadly, this can be achieved by modulating (or, alternatively expressed, altering) the concentration of Ca²⁺ in the secretory lysosomes and/or interfering in, or modulating, any one or more steps of the signalling pathway indicated above that influence the formation of the mature dense core secretory lysosome.

By increasing the size of the secretory lysosomes it is possible to increase NK cell functionality, and it is proposed that the same effect may be obtained by increasing the number of secretory lysosomes. The reverse is also the case, i.e. that NK cell functionality may be decreased by reducing the size of the secretory lysosomes, and it is proposed that the same effect may be obtained by reducing the number of secretory lysosomes. The inventors of the present application have developed an approach whereby the rates of lysosomal fission (i.e. lysosome division) and lysosomal fusion are altered so as to modulate the size and number of lysosomes in a target granular immune effector cell. This results in an alteration of the signalling capacity of the cell, as described herein.

Modulation of the content of the secretory lysosomes may also either up- or down-regulate NK cell functionality, as described herein. NK cell functionality may alternatively be increased or decreased by any other technique which modulates the signalling capacity of these secretory lysosomes. For instance, by increasing or decreasing the basal rate of uptake and sequestration of cytosolic Ca²⁺, the amount of Ca²⁺ influx into the cytosol required to activate NK cell functionality (degranulation and cytokine release) is affected, effectively increasing or reducing the threshold level of Ca²⁺ influx into the cytosol required to activate NK cell functionality.

A further method by which NK cell functionality may be modulated is by increasing the concentration of Ca²⁺ in the secretory lysosome. This can be achieved by the direct manipulation of the matrix of the secretory lysosomes, e.g. by manipulating serglycin and/or other polyanionic matrix components. Alteration of the structure of the secretory lysosome matrix can enhance (or reduce) lysosomal Ca²⁺ content, thus increasing (or reducing) the signalling capacity (i.e. signalling potential) of the secretory lysosome and the activity of the immune effector cell.

The modulation of NK cell functionality is seen not only at the level of cytotoxic (i.e. cell killing) activity, but also in relation to other aspects of effector cell function, for example cytokine production. It is accordingly proposed that the same methods may also be used to modulate the function of other granular immune effector cells, particularly T-cells, and including not only cytotoxic cells but also cells whose activity is primarily achieved by production and release of cytokines, such as T-helper cells and Treg cells.

Such methods of up- or down-regulating the function of granular immune effector cells may be employed in a number of contexts. In particular, the modulation of granular immune effector cell function may be useful for the in vivo treatment of subjects suffering from one or more conditions in which the up- or down-regulation of cytotoxic immune effector cell activity is beneficial to the subject. Methods whereby the activity of an immune effector cell is up-regulated may alternatively be employed ex vivo to granular immune effector cells to prepare cells for use in adoptive transfer therapy, and in vitro or ex vivo to modulate the activity of a granular immune effector cell. Such ex vivo and in vitro methods hold great promise for the enhancement of adoptive cell therapy. Currently, adoptive cell therapy is often seen to fail or to be ineffective due to the transfused immune cells achieving an insufficient level of activity for efficacy. Enhancement of the activity of cells for use in adoptive cell therapy using the methods of the invention can lead to increased treatment efficacy and a lower chance of treatment failure. Cells produced by the methods of the invention may be used in therapy for a number of conditions, including cancers, immunodeficiencies, infections and inflammatory disorders.

Modulation (i.e. the up-regulation or down-regulation) of the activity of a granular immune effector cell, in the context of the invention, can be achieved by applying to the cell an agent which modulates the size, content and/or number of secretory lysosomes in said cell, or otherwise modulates the signalling capacity of the secretory lysosomes.

In a first aspect the invention provides a method of preparing a granular immune effector cell for adoptive cell therapy, the method comprising up-regulating the activity of the granular immune effector cell by contacting the cell ex vivo with an agent which modulates the size, content and/or number of secretory lysosomes in the cell, or otherwise modulates the signalling capacity of the secretory lysosomes in the cell.

The effect on the activity of a granular immune effector cell of an agent which modulates the size, content and/or number of secretory lysosomes in said cell, or otherwise modulates the signalling capacity of the secretory lysosomes in said cell, may be enhanced by applying the agent in combination with a second species (i.e. a second agent) which affects signalling from an inhibitory receptor expressed on the surface of the granular immune effector cell, either by targeting the receptor directly or by targeting signalling pathways or cascades downstream of the receptor. Such a second species may be a ligand, or agonist of an inhibitory receptor expressed by the granular immune effector cell, or an activator of a signalling pathway downstream of said inhibitory receptor. Accordingly the “lysosome-modulating” agent of the invention (hereinafter “the first agent”) may optionally, or in some embodiments of the various aspects of the invention, be used in combination with such a second agent.

Thus, in another aspect, the invention provides an in vitro or ex vivo method of up-regulating the activity of a granular immune effector cell, the method comprising contacting the cell with an agent which modulates the size, content and/or number of secretory lysosomes in the cell, or otherwise modulates the signalling capacity of the secretory lysosomes in the cell, in combination with a ligand or agonist of an inhibitory receptor expressed by the cell and/or an activator of a signalling pathway downstream of the inhibitory receptor.

The invention also provides a cell or population of cells produced by the methods of the invention. In certain embodiments, the cell or population of cells of the invention is provided in the form of a pharmaceutical composition containing the cell or population of cells together with one or more pharmaceutically-acceptable diluents, carriers or excipients. Accordingly, the invention also provides a pharmaceutical composition comprising a cell or population of cells of the invention and one or more pharmaceutically-acceptable diluents, carriers or excipients.

In another aspect, the invention provides a cell, population of cells or pharmaceutical composition of the invention for use in therapy, preferably adoptive cell therapy. The invention also provides a method of treatment comprising administering a cell, population of cells or pharmaceutical composition of the invention to a subject, preferably wherein the treatment is adoptive cell therapy, preferably wherein the subject is human.

In another aspect, the invention provides a kit comprising a first agent which modulates the size, content and/or number of secretory lysosomes in a granular immune effector cell, or otherwise modulates the signalling capacity of the secretory lysosomes in a granular immune effector cell, and a second agent selected from (i) a ligand, agonist or antagonist of an inhibitory receptor expressed by a granular immune effector cell; or (ii) an activator or inhibitor of a signalling pathway downstream of such an inhibitory receptor. Preferably, the kit of the invention comprises a second agent selected from (i) a ligand or agonist of an inhibitory receptor expressed by a granular immune effector cell; or (ii) an activator of a signalling pathway downstream of said inhibitory receptor.

The kit of the invention may be used in up- or down-regulating the activity of granular immune effector cells. The first and second agents may be used separately, sequentially or simultaneously, and may be formulated together in the same composition or in separate compositions (e.g. in separate containers or in the same containers). The kit of the invention may be used for preparing a granular immune effector cell for adoptive cell therapy according to the method of the invention. The kit may also more generally be used in the in vitro or ex vivo method of up-regulating the activity of a granular immune effector cell according to the invention, of for producing a cell or population of cells of the invention.

The kit of the invention may also be used in up- or down-regulating the activity of granular immune effector cells for non-therapeutic purposes, e.g. for experimental purposes.

As noted above, as well as the ex vivo or in vitro uses presented above, the methods of the invention may also be used in vivo for therapeutic purposes.

According to such an aspect, the invention also provides an agent for use in up- or down-regulating the activity of a granular immune effector cell in therapy, wherein said agent modulates the size, content and/or number of secretory lysosomes in said cell, or otherwise modulates the signalling capacity of the secretory lysosomes.

This aspect of the invention also provides the use of an agent which modulates the size, content and/or number of secretory lysosomes in a granular immune effector cell, or otherwise modulates the signalling capacity of the secretory lysosomes in a granular immune effector cell, in the manufacture of a medicament for use in up- or down-regulating the activity of a granular immune effector cell.

The invention also provides a method of treatment, wherein the activity of a granular immune effector cell is up- or down-regulated, said method comprising administering to a subject an agent which modulates the size, content and/or number of secretory lysosomes in said cell, or which otherwise modulates the signalling capacity of the secretory lysosomes in said cell.

In certain embodiments, the agent for use in up- or down-regulating the activity of a granular immune effector cell in therapy may be provided in the form of a pharmaceutical composition containing the agent together with one or more pharmaceutically acceptable diluents, carriers or excipients.

Accordingly, the invention also provides pharmaceutical compositions comprising an agent which modulates the size, content and/or number of secretory lysosomes in a granular immune effector cell, or which otherwise modulates the signalling capacity of the secretory lysosomes in a granular immune effector cell, together with one or more pharmaceutically acceptable diluents, carriers or excipients, for use in up- or down-regulating the activity of a granular immune effector cell.

In another aspect the invention also provides a product comprising a first agent as defined herein and a second agent selected from a ligand, agonist or antagonist of an inhibitory receptor expressed by a granular immune effector cell or an activator or inhibitor of a signalling pathway downstream of said inhibitory receptor, as defined herein, as a combined preparation for simultaneous, separate or sequential use in up- or down-regulating the activity of granular immune effector cells in therapy.

The agent which modulates the signalling capacity of the secretory lysosomes of the granular immune effector cell according to the invention is an agent which modulates the size and/or content and/or number of the secretory lysosomes in the cell. In particular embodiments of the various aspects of invention, the agent modulates the size and/or content of the secretory lysosomes in the cell, particularly the size. Further in particular, the agent modulates the secretory lysosomes which are already present in the cell, i.e. existing lysosomes. This may include modulating the number of lysosomes by modulating the fusion and/or fission of existing lysosomes.

In particular embodiments the agent does not induce lysosome biogenesis. By lysosomal biogenesis as referred to herein is meant de novo lysosome formation, as is believed to occur by the fusion of a late endosome with a Golgi apparatus-derived vesicle containing lysosomal enzymes. The multiplication of lysosomes by the fission of existing lysosomes is considered herein to be a distinct process and is not encompassed by the term “lysosome biogenesis”. In particular embodiments the agent is not a cytokine which induces lysosome biogenesis. In a particular embodiment the agent is not IL-15 In other embodiments the agent is not an mTOR inhibitor, e.g. it is not rapamycin.

It will be understood and well known to a person skilled in the art in this field that up- or down-regulation of immune effector cell activity may be of use in a number of therapeutic contexts, i.e. that there are a number of clinical conditions (which term is used broadly herein to include any medical condition, disease or disorder) that may be responsive to, or which may benefit from, an up- or down-regulation of immune effector activity, and that any such condition may be treated or prevented according to the present invention. Thus, for example, certain conditions may be associated with unwanted or elevated immune effector cell activity and may accordingly benefit from reducing (i.e. decreasing or dampening) this activity, e.g. autoimmune disorders, allergic reactions, inflammation etc. Such conditions may also be treated by up-regulating the activity of regulatory immune cells, which in turn down-regulate the activity of other aspects of the immune system and the immune response. Other conditions may be associated with immunosuppression (e.g. unwanted or increased immune suppression, or immune evasion, such as may occur with certain cancers etc.) and may benefit from increasing immune effector cell activity. Other conditions, notably cancer and infections may benefit from an increase in immune effector cell activity, including increased immune cytotoxic cell activity to abrogate unwanted or deleterious target cells (e.g. cancer cells or infected cells). It will thus be seen that the invention may be defined as up- or down-regulating a cell-based immune response (i.e. an immune response mediated by an immune effector cell, or more particularly a granular immune effector cell). The cell-based immune response may be regulated by up- or down-regulating the immune response in vivo, i.e. by the administration of a first agent according to the invention directly to a subject, or by the administration to a subject of immune cells in order to supplement the subject's natural immune response, i.e. by adoptive cell therapy. Cells to be administered to a subject via adoptive cell therapy may be prepared using the in vitro or ex vivo methods of the invention in order to enhance their activity and thus increase the efficacy of the adoptive cell therapy.

The agent of the invention is able to up- or down-regulate the activity of a granular immune effector cell by modulating the size, content and/or number of secretory lysosomes in the granular immune effector cell, or by otherwise modulating the signalling capacity of the secretory lysosomes in the granular immune effector cell.

An “immune effector cell” is any cell of the immune system that has one or more effector functions (e.g. cytotoxic cell killing activity, secretion of cytokines, chemokines or other molecules, induction of antibody-dependent cell-mediated cytotoxicity (ADCC), regulatory activity etc.). A granular immune effector cell is one which contains secretory lysosomes. As used herein, the term “granular immune effector cell” is not limited to effector cells containing granules in the commonly used sense (in which “granules” refer to secretory lysosomes comprising cytotoxins).

Representative granular immune effector cells thus include T-cells, in particular CTLs (CD8⁺ T-cells), helper T-cells (Tₕ cells; CD4⁺ T-cells; HTLs), natural killer T-cells (NKT cells) and regulatory T-cells (Tregs). Many subspecies of HTLs may be useful in the invention, including Tₕ1 cells, Tₕ2 cells and Tₕ17 cells. Innate lymphoid cells (ILCs) are also of particular interest in the invention. ILCs are immune cells of lymphoid lineage but which do not function in an antigen-specific manner. ILCs useful in the present invention include ILC1, ILC2, ILC3 and NK cells, particularly NK cells. Other granular immune effector cells include neutrophils and macrophages. Immune effector cells also include progenitors of effector cells, wherein such progenitor cells can be induced to differentiate into immune effector cells in vivo or in vitro. Such progenitors include stem cells, and in the case of the in vitro or ex vivo uses of the invention this includes induced pluripotent stem cells.

In one embodiment the immune effector cell is a cytotoxic immune effector cell and in another embodiment T-cells, particularly CD8⁺ T-cells, and NK cells represent preferred immune effector cells according to the invention. CD4⁺ T-cells and Treg cells are also preferred cells for use in the invention.

The term “NK cell” refers to a large granular lymphocyte, being a cytotoxic lymphocyte derived from the common lymphoid progenitor which does not naturally comprise an antigen-specific receptor (e.g. a T-cell receptor or a B-cell receptor). The term as used herein thus includes any known NK cell or any NK-like cell or any cell having the characteristics of an NK cell.

In the case of the in vivo medical therapies disclosed herein, the immune effector cells will of course be the endogenous native cells of the subject under treatment. In the case of the in vitro or ex vivo uses, the immune effector cells may be primary cells, for example they may isolated from a subject. Alternatively, they may be cultured or modified cells (e.g. genetically modified or engineered), or they may be cells of a cell line.

A secretory lysosome may alternatively be referred to as a lysosome-related organelle. It may be defined as a lysosome-like structure in the cell which is able to secrete one or more components out of the cell. It is accordingly, in other terms, a secretory vesicle.

As noted above, secretory lysosomes are present in all granular immune effector cells and generally contain components which are able to degrade cellular material, or indeed in some cases cells (i.e. which are cytotoxic). By “content” of secretory lysosomes is meant any of the components which are contained in a secretory lysosome. Generally, this will include enzymes, including proteases, and particularly granzymes (e.g. granzyme B in the context of cytotoxic immune effector cells). Other components (particularly in the case of cytotoxic immune effector cells) may include pore-forming agents, e.g. perforin, or other agents which are capable of disrupting a cell membrane. Accordingly, in certain embodiments the secretory lysosome of the cell may contain granzyme B. However, the invention is not limited to such immune effector cells, and it is important to note that the activity (or functional response) of the immune effector cell is not necessarily related to the content of the secretory lysosome (for example in the case of Tₕ, Treg and certain ILCs the activity or functional response is unrelated to the lysosome contents).

The signalling capacity of the secretory lysosomes may be defined more precisely as the capacity of the lysosome to propagate signalling from surface receptors on the cell, or more particularly from the cell surface receptors to the ER or nucleus of the cell. Thus the secretory lysosome may mediate signalling within the cell, including in particular in the context of the activity of the immune effector cell, e.g. the signals involved in activation or stimulation of the cell, or the mechanisms involved in achieving the activity (i.e. functional response) of the cell. Particularly the signalling by the lysosomes may involve, or comprise, changes in Ca²⁺ flux in the cell, e.g. efflux of Ca²⁺ from the lysosome into the cytosol. Various different signalling pathways may be involved, as described further below. Thus, the signalling capacity of the cell may include pathways up- and/or down-stream of the secretory lysosome. It will be understood that such up- and/or down-stream pathways which may be modulated by the agent are pathways which are connected to the lysosome.

Immune effector cell activity may be any activity possessed or demonstrated by an immune effector cell, including notably cytotoxic activity, but also an activity in producing cytokines (which term is used broadly herein to include all cytokines and chemokines), and/or other regulatory or signalling molecules. Thus, effector cell activity may be any functional response of an immune effector cell and includes regulatory activity, or any activity in potentiating, assisting, or reducing the activity of other cells.

In one embodiment of the invention, the “activity” of a granular immune effector cell refers to the cytotoxic activity of the granular immune effector cell. This embodiment is clearly only applicable to cytotoxic cells. By the “cytotoxic activity” of a granular immune effector cell is meant its release of cytotoxins in the process of degranulation. Increased, or up-regulated, cytotoxic activity of an immune effector cell may mean in certain embodiments that the amount of cytotoxins released by the cell during degranulation is increased. Such a result may for instance be achieved by increasing the number and/or size of the lysosome, or by increasing the cytotoxin content (i.e. the amount and/or nature or composition of the cytotoxins) of the secretory lysosomes present in the cell. The cytotoxins may be any used by cytotoxic lymphocytes, particularly granzyme B, perforin and granulysin, though other cytotoxins (e.g. granzyme A) may also be present. Alternatively, or additionally, cytotoxic activity of an immune effector cell may be increased by promoting degranulation, by for instance effectively lowering the threshold for the amount of Ca²⁺ influx into the cytosol required to stimulate degranulation. Any other method of altering signalling in the cell to promote degranulation may also be used.

Thus an increase in cytotoxic activity may be achieved by increasing or promoting cytotoxin release by a cytotoxic immune effector cell. The reverse is also true, i.e. that the cytotoxic activity of a cytotoxic immune effector cell may be decreased, or down-regulated, by reducing the size, number or cytotoxin content of the secretory lysosomes present in the cell, or by repressing degranulation, by any method known in the art.

In another embodiment, the activity of a granular immune effector cell refers to cytokine production by the cell. Up-regulation of cytokine production may mean that the cell produces an increased amount, or level, of cytokine, and/or releases larger amounts of cytokine. Alternatively, or additionally, up-regulation of cytokine production may mean that cytokine production is stimulated such that the conditions required for induction of cytokine production are made less stringent, e.g. by effectively lowering the threshold of e.g. Ca²⁺ influx into the cytosol required for cytokine production and release. The reverse is also true, i.e. down-regulation of cytokine production may refer to the reduction and/or repression of cytokine production. The cytokines produced by the granular immune effector cell may include interferons, such as IFNα, IFNβ or IFNγ, transforming growth factors, such as TGFβ, tumour necrosis factors, such as TNFα or TNFβ, interleukins, such as IL-2, IL-3, IL-4, IL-8, IL-9, IL-10, IL-13, IL-17, IL-22 and IL-23 and chemokines such as CXCR1, CXCR2, CXCR3, CXCR4, and CX3CR1. The particular cytokines produced by a certain type of granular immune effector cell are known to the skilled person and can be readily found in textbooks etc.

The activity of a granular immune effector cell may be up- or down-regulated by modulating the content of the secretory lysosomes present in the cell. As mentioned above, in the context of a cytotoxic cell the content of the secretory lysosomes modulated to up- or down-regulate the activity of the cell may be the cytotoxin content of cytolytic granules. However, the content of the secretory lysosomes, as defined herein, is by no means restricted to cytotoxins. In particular, the content of a secretory lysosome may refer to the matrix which supports Ca²⁺ loading of the lysosome. This encompasses all proteins which regulate the Ca²⁺ homeostasis (e.g. concentration in, influx to and efflux from the lysosome). By increasing the lysosomal content of this matrix the Ca²⁺ capacity of the lysosomes can be increased (and vice-versa), which thus influences Ca²⁺ uptake and release by the lysosome. Examples of matrix components include serglycin (which may be modified by chondroitin sulphate) and other polyanionic matrix components. Enzymes which catalyse the formation of secretory lysosome matrix components include Carbohydrate (Chondroitin 4) Sulfotransferase 11, Carbohydrate (Chondroitin 4) Sulfotransferase 12 and N-Deacetylase/N-Sulfotransferase-2 Putative immune effector cell secretory lysosome matrix components include chromogranins and von Willebrand factor.

The content of a secretory lysosome as defined herein also refers to the pH of the lysosome, thus encompassing both the H⁺ concentration of the lysosome (i.e. its acidity) and proteins involved in proton homeostasis. The pH of a lysosome is well known to affect Ca²⁺ homeostasis by the lysosome, including Ca²⁺ uptake and release. Proteins involved in proton homeostasis include the V-ATPase. The term “V-ATPase” as used herein encompasses all subunits of the V-ATPase, both of the V₀ and V₁ domains, including subunits A, B, C, D, E, F G, H, a, d, c, c′, c″ and e.

The agent for use in up-regulating the activity of a granular immune effector cell may be an agent which promotes homotypic lysosomal fusion (i.e. promotes the fusion of lysosomes to each other) and/or reversibly inhibits lysosomal fusion to the cell membrane, promoting retention of the secretory lysosomes in the cell. In a cytotoxic immune effector cell, the promotion of secretory lysosome retention inhibits degranulation. Alternatively, the agent for use in up-regulating the activity of a granular immune effector cell may be an agent which prevents lysosomal fission, i.e. an agent which prevents division of secretory lysosomes. Such agents cause an increase in the size of secretory vesicles, thus increasing the level of activity of the immune effector cell. In particular, in a cytotoxic immune effector cell the reversible inhibition of fusion of secretory lysosomes to the cell membrane, and/or the prevention of lysosome fission, cause a build-up of cytotoxic potential in the cell, meaning that when the cell is activated to kill a target cell its cytotoxic activity is significantly increased.

Agents which promote homotypic lysosomal fusion, prevent lysosomal fission and/or reversibly inhibit lysosomal fusion to the cell membrane include commercially available PIKfyve inhibitors, such as vacuolin-1 (available from Santa Cruz Biotechnology), YM201636 (available from Cayman Chemical, Apilimod (available from Cayman Chemical) and/or APY0201 (available from Tebu-Bio). Such inhibitors may be used individually or in combination. PIKfyve inhibitors are known to drive the formation of giant vacuoles in cells. Other agents which inhibit the signalling pathway which activates PIKfyve, or effector proteins of the signalling pathway downstream of PIKfyve, can similarly be used to promote homotypic lysosomal fusion or prevent lysosomal fission. For instance, inhibitors of Akt, TRPML1, TRPML2, transient receptor potential cation channel melastatin 2 (TRPM2), a ryanodine receptor (RyR) or two pore channel 1 (TPC1) or TPC2 may be used. Inhibitors of other proteins including CD38 and CD31 may also be used to up-regulate granular immune effector cell activity. Notably, the CD38-initiated signalling pathway is important for NK cell effector responses, acting through ADPr (adenosine diphosphate ribose) which activates TRPM2. An agent which inhibits CD38 and/or TRPM2 may thus be used in the ex vivo and in vitro methods of the invention, but may not be suitable for use as an in vivo therapeutic. Moreover, when an agent which inhibits CD38, TRPM2, TRPML1, TRPML2, TPC1, TPC2 or RyR is used in an ex vivo or in vitro method of the invention in the preparation of cells for adoptive cell therapy, it is preferable that the agent is only transiently applied to the immune effector cell. Such an agent can thus be used to induce a granular phenotype in the cell (i.e. to promote lysosomal fusion and/or prevent lysosomal fission), but should be removed from the cells prior to administration to a subject. Other agents with the same or similar effects on lysosomal fusion and retention may also or alternatively be used. Such agents may be easily identified by screening or may be rationally designed. Tests for determining or assessing lysosomal fusion activity etc. are readily available in the art. (See for example the experiments described in Example 1 below.)

In one embodiment where the activity of immune effector cells is modulated to treat cancer, the agent is not Apilimod. In particular, in such an embodiment when the activity of immune effector cells is modulated in vivo to treat cancer (i.e. in the context of a medical use of the agent by its administration to a subject), the agent is not Apilimod.

An agent with the reverse effect, i.e. an agent which promotes lysosomal fusion to the cell membrane and thus reduces lysosomal retention in immune effector cells may be used to down-regulate the activity of a granular immune effector cell.

In another embodiment, the agent for use in up-regulating the activity of a granular immune effector cell is an agent which increases the capacity of the secretory lysosomes to channel or buffer calcium responses, i.e. an agent which increases the capacity of the secretory lysosomes to initiate Ca²⁺ signalling cascades within the cell. Such an agent may be an agent which increases the cytosolic level of Ca²⁺ in the cell. This has the effect of reducing the amount of Ca²⁺ influx required to activate the cell functions, e.g. to stimulate degranulation or cytokine production, effectively lowering the threshold Ca²⁺ concentration for immune cell activation. Cytosolic Ca²⁺ levels may be increased by increasing flow of Ca²⁺ into the cytosol (i.e. increased influx) and/or by decreasing uptake of Ca²⁺ from the cytosol (i.e. decreased efflux). Several mechanisms of increasing Ca²⁺ levels in the cytosol of the immune effector cell exist. One mechanism of increasing cytosolic Ca²⁺ levels is by depleting the endoplasmic reticulum (ER) Ca²⁺ stores. This may be done by blocking Ca²⁺ uptake by the ER, particularly SERCA-mediated Ca²⁺ uptake. In an exemplary embodiment, SERCA-mediated Ca²⁺ uptake to the ER is blocked by thapsigargin (available from Sigma-Aldrich). However, any other agent which blocks Ca²⁺ uptake by the ER may be used, for instance cyclopiazonic acid. Again, such agents may be easily identified by screening or rationally designed.

Another mechanism of increasing the cytosolic Ca²⁺ level is to block Ca²⁺ uptake by the secretory lysosome itself, and/or more generally by the acidic compartments of the cell. This may be done, for example, by blocking cholesterol production, which leads to an accumulation of sphingosine in the secretory lysosome, which in turn blocks Ca²⁺ uptake thereinto. In an exemplary embodiment Ca²⁺ uptake by the secretory lysosome is blocked using the inhibitor of cholesterol production U18666a (available from Cayman Chemical). Again, other such agents may be easily identified by screening or rationally designed.

Various other techniques whereby cytosolic Ca²⁺ levels can be increased are also known. This may be achieved by increasing flux of Ca²⁺ into the cytosol from other cellular compartments, including the ER or acidic compartments. For instance, cytosolic Ca²⁺ levels can also be increased by activating CD38, which produces Ca²⁺ mobilising second messengers, including NAADP, ADPR and cyclic ADPR (cADPR). In certain embodiments of the invention the agent used to increase cytosolic Ca²⁺ levels is not NAADP, ADPR or cADPR.

In certain embodiments, the agent may promote the release from and/or block the uptake of Ca²⁺ by the acidic compartment(s) of the cell and in particular from and/or by the secretory lysosomes themselves.

In a preferred embodiment of the invention, at least two techniques for increasing cytotosolic Ca²⁺ levels are used together, for instance blocking Ca²⁺ uptake by both the ER and secretory lysosomes. For instance, the agent used to increase cytosolic Ca²⁺ levels may comprise both thapsigargin and U18666a. However, agents which increase cytosolic Ca²⁺ levels are not suitable for use in the ex vivo or in vitro methods of the invention. Accordingly, for the ex vivo/in vitro methods described herein, in certain embodiments, the agent is not an agent which increases cytosolic Ca²⁺, but rather is an agent which increases the level of Ca²⁺ in the secretory lysosomes, or more generally in the acidic compartments of the cell, as discussed below.

Conversely, the activity of a granular immune effector cell may be down-regulated by reducing the levels of cytosolic Ca²⁺, for instance by enhancing Ca²⁺ uptake by the ER or suchlike.

The agent which increases the capacity of the secretory lysosomes to channel or buffer calcium responses may alternatively be an agent which increases the level of Ca²⁺ in the secretory lysosomes of the immune effector cell, thus enhancing their signalling potential. This may be achieved by altering the matrix of the secretory lysosomes.

The agent for use in reducing granular immune effector cell function may be a lysosomotropic agent, which is defined herein as an agent which causes osmotic disruption of the lysosomal membrane. Such agents cause depletion of Ca²⁺ stores within secretory lysosomes, and consequently result in the loss of Ca²⁺ derived signalling from the acidic compartment. In an exemplary embodiment of the invention, the lysosomotropic agent for use in down-regulating granular immune effector cell function is one or more of Gly-Phe-β-naphthylamide (GPN) (available from Cayman Chemicals), mefloquine (available from Sigma-Aldrich) or siramesine (available from Sigma-Aldrich). GPN is a substrate of cathepsin C that accumulates within the lysosome. Hydrolysis of GPN by Cathepsin C produces fragments which do not easily diffuse through the lysosomal membrane, leading to a loss of lysosome membrane integrity. In an embodiment, where the activity of immune effector cells is modulated in vivo to treat an inflammatory disorder, the agent is not mefloquine.

The agent used to up- or down-regulate the activity of a granular immune effector cell may be a small molecule or suchlike, as described above. In a further embodiment of the invention, the agent used to up- or down-regulate the activity of a granular immune effector cell does so by altering gene expression patterns in the cell. Such an agent modulates gene expression. An agent which modulates gene expression may activate, inactivate, increase or decrease expression of a gene. By activate is meant “switching on” expression of a gene which would not otherwise be expressed under the same conditions. By increase is meant that the agent causes a higher level of expression of a gene which would otherwise be expressed at a lower level (i.e. over-expression of a gene). By inactivate is meant the opposite of activate (i.e. “switching off” expression of a gene which would otherwise be expressed) and by decrease is meant the opposite of increase (i.e. the agent causes a lower level of expression of a gene which would otherwise be expressed at a higher level). Inactivation of a gene may be reversible (e.g. its transcription or translation may be specifically inhibited), or it may be irreversible (e.g. the genome of the cell may be edited to delete the gene). Increasing or reducing expression of particular genes may be performed by knockdown of gene expression, or modulation of transcription factor expression activity. Alternatively, genome editing may be used to insert particular promoters or regulatory elements up- or downstream of the target gene, as appropriate, in order to increase or decrease gene expression, or to allow gene expression to be controlled. For instance, a weak or strong promoter may be inserted upstream of a target gene to reduce or increase expression, respectively. Alternatively, an inducible promoter may be inserted upstream of the target gene to allow tight regulation of the level of expression of the target gene. Thus, in an embodiment of the invention, the agent used to up- or down-regulate the activity of the granular immune effector cell alters gene expression in the cell (i.e. activates, inactivates, increases or decreases expression of one or more target genes). Such an agent may be an RNA molecule which mediates RNAi. RNAi is a process well known and easily employed by those skilled in the art which may be used to knockdown or inhibit (i.e. decrease or inactivate) gene expression. The skilled person will be able to design an appropriate RNA molecule for use in RNAi targeting a specific gene of interest.

Such an agent may alternatively be an agent for use in gene editing. Several gene editing techniques are known in the art, but most preferably the CRISPR/Cas9 technique may be used. This process is well known to those skilled in the art. The CRISPR/Cas9 technique employs the use of a single guide RNA (sgRNA) and a Cas9 nuclease. The skilled person will be able to design an appropriate sgRNA molecule for use in CRISPR/Cas9 editing (e.g. deletion) of a specific gene of interest.

In another alternative, such an agent may be a compound which alters the expression level of one or more target genes by modulating the level of transcription of the gene(s) without altering the genotype of the cell (i.e. without performing gene editing). Mechanisms by which transcription may be so altered include activating, deactivating or mimicking signalling pathways in a cell, and up-regulating, down-regulating, activating or inactivating transcription factors (at the level of transcription, translation or post-translationally). Transcription factor activity may be modulated at the post-transcriptional level by e.g. stimulating their phosphorylation/dephosphorylation, or targeting them for degradation by modulating the ubiquitination activity of the cell. Such a compound may be a small molecule, such as a pharmaceutical, or a small biological molecule such as a peptide or a signalling molecule such as cAMP or cGMP. Such a compound may alternatively be a macromolecule such as a protein.

The gene(s) targeted in this embodiment of the invention may be any gene(s) which affect the activity of a granular immune effector cell. In particular, the gene may be part of a signalling pathway upstream of the secretory lysosome. Thus the agent which activates, inactivates, increases or decreases expression of one or more target genes preferably modulates the expression of one or more signalling pathways (or a component thereof, e.g. a signalling molecule) upstream of the secretory lysosome. The modulation of the expression of one or more signalling pathways (or components thereof) upstream of the secretory lysosome may be in the form of activation, inactivation, increasing or decreasing of expression of the pathway. Preferably, one or more of the following genes may be targeted to modulate expression of one or more signalling pathways upstream of the secretory lysosome: CD38, CD31, TRPM2, TRPML1, TRPML2, RyR, TPC2 and PIKFYVE. Reducing or inactivating expression of CD38, CD31, TRPM2, TRPML1, TRPML2, RyR, TPC2, and/or PIKFYVE would up-regulate immune effector cell activity, and conversely increasing or activating expression of CD38, CD31, TRPM2, TRPML1, TRPML2, RyR, TPC2, and/or PIKFYVE would down-regulate immune effector cell activity.

Alternatively, the gene(s) targeted in this embodiment of the invention may be a gene which encodes a component of the lysosome matrix, or which contributes to lysosome matrix assembly or formation. In particular, one or more of the following genes may be targeted to alter lysosome assembly: SRGN, CHST11, CHST12, NDST2, CST7, GNPTAB and M6PR. In particular, genes which encode proteins with calcium binding capacity may be targeted. Increasing or activating expression of SRGN, CHST11, CHST12, NDST2, CST7, GNPTAB and/or M6PR would up-regulate immune effector cell activity, and conversely decreasing or inactivating expression of SRGN, CHST11, CHST12, NDST2, CST7, GNPTAB and/or M6PR would down-regulate immune effector cell activity. Expressing chromogranin genes in immune effector cells may also alter secretory lysosome assembly in immune effector cells, in particular to up-regulate immune effector cell activity. Chromogranins are key components of secretory lysosomes in neuroendocrine cells and it is proposed that their expression in immune effector cells will increase secretory lysosome activity. Accordingly, activation of expression of a chromogranin gene, in particular CHGA or CHGB, may up-regulate immune effector cell activity. Expressing the gene encoding von Willebrand factor (vWF) in immune effector cells may also alter secretory lysosome assembly in immune effector cells to up-regulate immune effector cell activity. Chromogranin genes and VWF may be expressed in immune effector cells either by specific induction of expression of the chromosomal genes, or by exogenous expression of additional copies of the genes introduced into the immune effector cell, e.g. on a vector.

In particular aspects of this embodiment, immune effector cell activity is up-regulated by increasing expression of SRGN (serglycin), CHST11 (Carbohydrate sulfotransferase 11) and/or CHST12 (Carbohydrate sulfotransferase 12) using the protein activin, the second messenger cAMP or the pharmaceutical product lenalidomide. Modulating the expression of genes such as SRGN, the products of which localise to secretory lysosomes or form constituent parts of the lysosome matrix, can thus modulate the content of secretory lysosomes as defined herein. In preferred aspects of the invention the expression of genes of the secretory lysosome Ca²⁺-loading matrix is modulated, or the expression of genes which regulate lysosomal pH is modulated.

The skilled person will readily be able to identify other appropriate genes and/or signalling pathways, the expression of which can be modulated to up- or down-regulate the activity of a granular immune effector cell, and in particular which affect, or modulate, the signalling by the secretory lysosome.

The agent of the invention may target the granular immune effector cell at any one of three stages: at the priming stage, by affecting the transcriptional and epigenetic functional programming that happens during effector cell development; during functional tuning during cellular homeostasis, e.g. by manipulation of cell-to-cell interactions (in the context of an NK cell, this functional tuning is equivalent to education); and at the effector stage, by targeted boosting of effector function during target interaction. All of these possibilities may be used in vivo (i.e. by the agent for use in up- or down-regulating the activity of a granular immune effector cell in therapy, and in the method of treatment wherein the activity of a granular immune effector cell is up- or down-regulated). The agent for use in therapy or which is administered to a subject within a method of treatment may therefore target the granular immune effector cell at the priming stage (during effector cell development), during cellular homeostasis or at the effector stage. In the ex vivo and in vitro methods of the invention, the agent which up-regulates granular immune effector cell activity may target the granular immune effector cell at the priming stage, during effector cell development, or during cellular homeostasis (i.e. the agent may manipulate or modulate cell-to-cell interactions).

When an immune effector cell is targeted at the priming stage, the accumulation of secretory lysosomes in the cell may be promoted (in the context of a cytotoxic immune effector cell these secretory lysosomes may be or include cytotoxic granules). The accumulation of secretory lysosomes in the cell up-regulates the activity or potential activity of the immune effector cell. When an immune effector cell is targeted at the effector stage, this boosts effector function when it interacts with a target. In the case that the immune effector cell is a cytotoxic immune cell, this boosting of effector function increases cytotoxic killing of target cells by the immune cell.

As mentioned above, the agent for use in the invention (i.e. the first agent) may advantageously be used in combination with a second agent which affects signalling from an inhibitory receptor expressed on the surface of the granular immune effector cell. Such a second agent may either target the receptor directly or target signalling pathways or cascades downstream of the receptor. Such an agent may be a ligand, agonist, or antagonist of an inhibitory receptor expressed by the target immune effector cell or an activator or inhibitor of a signalling pathway downstream of said inhibitory receptor. The term agonist as used herein includes reversible agonists, for example binding molecules such as antibodies, which may be released from the inhibitory receptor. In the case that the second agent is a ligand or agonist of an inhibitory receptor, or an activator of a pathway downstream of such an inhibitory receptor, application of this species to an NK cell has the effect of mimicking education of the NK cell, which would occur naturally by interactions between its inhibitory receptors and ligands on “self” cells (i.e. ligands expressed on or by other cells of the same individual organism, e.g. the same person).

As discussed above, education of an NK cell is known to enhance its activity, particularly its cytotoxic activity. Thus by activating inhibitory signalling pathways in a granular immune effector cell, its activity may paradoxically be increased. Conversely, use of a second species which inactivates inhibitory pathways of a granular immune effector cell, such as an antagonist of an inhibitory receptor or an inhibitor of a signalling pathway downstream of an inhibitory receptor, reduces the activity of the cell. Furthermore, ligands/agonists or antagonists of an inhibitory receptor may be used in combination with activators or inhibitors (respectively) of a signalling pathway downstream of such an inhibitory receptor, in order to further increase the effectiveness of the agents.

Thus, the effect of an agent for use in up-regulating the activity of a granular immune effector cell may be enhanced by use of the agent in combination with a ligand or agonist of an inhibitory receptor expressed on the cell, and/or an activator of a pathway downstream of such an inhibitory receptor. Similarly, the effect of an agent for use in down-regulating the activity of a granular immune effector cell can be enhanced by use of the agent in combination with an antagonist of an inhibitory receptor expressed on the cell and/or an inhibitor of a signalling pathway downstream of such an inhibitory receptor.

In the method of the invention for preparing a granular immune effector cell for adoptive cell therapy, the agent which up-regulates the activity of the immune effector cell may be used in combination with a ligand or agonist of an inhibitory receptor expressed on the cell, and/or an activator of a pathway downstream of such an inhibitory receptor. In the in vitro or ex vivo method of the invention for up-regulating the activity of a granular immune effector cell, the agent which up-regulates the activity of the immune effector cell is used in combination with a ligand or agonist of an inhibitory receptor expressed on the cell, and/or an activator of a pathway downstream of such an inhibitory receptor. In the in vivo methods of the invention in which a first agent is used to up- or down-regulate the activity of a granular immune effector cell in therapy, the first agent may be used in combination with a second agent which is a ligand, agonist, or antagonist of an inhibitory receptor expressed by the target immune effector cell or an activator or inhibitor of a signalling pathway downstream of said inhibitory receptor, as appropriate. The purpose of the second agent (ligand or agonist of an inhibitory receptor and/or activator of a downstream signalling pathway) is to increase the functional potential brought on by the modulation of the secretory lysosomes (i.e. by the first agent, which increases the activity of the immune effector cell).

The skilled person will be aware of the identities of the inhibitory receptors expressed on particular types of granular immune effector cell. In preferred embodiments of the invention, the inhibitory receptor expressed on the surface of the immune effector cell is selected from the group consisting of: Killer-Cell Immunoglobulin-like Receptors (KIRs), Programmed Cell Death Protein 1 (PD-1), T-Cell Immunoreceptor with Ig and ITIM Domains (TIGIT), T-Cell Immunoglobulin and Mucin-Domain containing-3 (TIM-3) and NKG2A. KIRs are inhibitory receptors expressed by NK cells and a minority of T-cells; PD-1 is expressed by T-cells; TIGIT is expressed by some T-cells and some NK cells; TIM-3 is expressed by T-cells; NKG2A is expressed by NK cells and some T-cells.

The agonist or antagonist of an inhibitory receptor may be any suitable species known in the art, e.g. it may be a small molecule, a protein or any other known agonist or antagonist. Preferably though, the agonist of an inhibitory receptor is an antibody. The term “antibody” is used broadly herein to refer to any type of antibody, or any antibody fragment or derivative. For example, the antibody may be polyclonal or monoclonal. The antibody may be of a single specificity. The antibody may be of any convenient or desired species, class or sub-type. Furthermore, the antibody may be natural, derivatised or synthetic. The term antibody as used herein thus includes all types of antibody molecules and antibody fragments.

More particularly the “antibody” may be:
(a) of any of the various classes or subclasses of immunoglobulin e.g. IgG, IgA, IgM, IgD or IgE derived from any animal e.g. any of the animals conventionally used e.g. sheep, rabbits, goats, or mice or egg yolk;
(b) a monoclonal or polyclonal antibody;
(c) an intact antibody or a fragment of an antibody, monoclonal or polyclonal, the fragment being one of those which contains the binding region of the antibody, e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. An Fv molecule may be defined as a fragment containing the variable region of the light chain and the variable region of the heavy chain expressed as two chains;
(d) an antibody produced or modified by recombinant DNA or other synthetic techniques, including monoclonal antibodies, fragments of antibodies, humanised antibodies, chimeric antibodies, or synthetically made or altered antibody-like structures. Also included are functional derivatives or “equivalents” of antibodies e.g. single chain antibodies. A single chain antibody may be defined as a genetically engineered molecule containing the variable region of the light chain, the variable region of the heavy chain, linked by a suitable polypeptide linker as a fused single chain molecule (e.g. an scFv). Also included are single chain (Sv) intrabodies.

Methods of making such antibody fragments and synthetic and derivatised antibodies are well known in the art. Also included are antibody fragments containing the complementarity-determining regions (CDRs) or hypervariable regions of the antibodies. These may be defined as the region comprising the amino acid sequences on the light and heavy chains of an antibody which form the three dimensional loop structure that contributes to the formation of the antigen binding site. CDRs may be used to generate CDR-grafted antibodies. As used herein “CDR grafted” defines an antibody having an amino acid sequence in which at least parts of one or more sequences in the light and/or variable domains have been replaced by analogous parts of CDR sequences from an antibody having a different binding specificity for a given antigen. One of skill in the art can readily produce such CDR grafted antibodies using methods well known in the art.

A chimeric antibody may be prepared by combining the variable domain of an antibody of one species with the constant regions of an antibody derived from a different species.

In particular embodiments, the agonist of an inhibitory receptor is a reversible agonist, such as releasable antibody, small molecule or suchlike. Use of such a reversible agonist enables the simulation of checkpoint inhibition-checkpoint reversal. Checkpoint inhibition-checkpoint reversal essentially means that development/activation of an immune effector cell is blocked at an immune checkpoint, and the blockade then reversed to allow the cell to proceed through the checkpoint. In the context of the invention, the development/activation of an immune cell may be blocked at an immune checkpoint while it is primed (i.e. while the agent of the invention is applied to up- or down-regulate the activity of the cell). Following the contacting of the immune cell with the agent the blockade of the immune checkpoint can then be reversed. Reversal can be achieved by e.g. competition, using for example a nanobody or suchlike, or by cleavage or dissociation of the reversible agonist. A reversible ligand, antagonist, activator or inhibitor may be similarly used.

The activator or inhibitor of a signalling pathway downstream of an inhibitory receptor may be an agonist or antagonist of a protein in such a signalling pathway, which can thus activate or inhibit signalling through the pathway. Preferred examples of proteins from signalling pathways downstream of inhibitory receptors which may be targeted in this invention include Akt, PI3K, Syk, Vav, PLC-g1, PLC-g2, LAT, SHP-1, c-Cbl, Cbl-b, and c-Abl. The agonist or antagonist of proteins such as these is preferably a small molecule, though can be any other suitable species known in the art. In particular, activation of SHP-1, c-Cbl, Cbl-b or c-Abl will prevent lysosomal fission and thus up-regulate the activity of the granular immune effector cell; conversely their inhibition will promote lysosomal fission and thus down-regulate the activity of the granular immune effector cell. Inhibition of Akt, PI3K, Vav, Syk, PLC-g1, PLC-g2 or LAT will prevent lysosomal fission and thus up-regulate the activity of the granular immune effector cell; conversely their activation will promote lysosomal fission and thus down-regulate the activity of the granular immune effector cell.

Accordingly, in the ex vivo and in vitro methods of the invention the first agent which up-regulates the activity of the granular immune effector cell may be used in combination with an activator, e.g. an agonist, of SHP-1, c-Cbl, Cbl-b or c-Abl and/or an inhibitor, e.g. an antagonist, of Akt, Vav, Syk, PI3K, PLC-g1, PLC-g2 or LAT.

In a further aspect the agent of the invention (i.e. the first agent) is used in combination with a stimulator of one or both of the paired receptors CD94/NKG2C (activating receptor) and CD94/NKG2A (inhibitory receptor) to produce cells which are NKG2C-positive and NKG2A-negative, i.e. according to the teachings of WO 2014/037422. The stimulator will thus be a ligand or agonist of one or both of the receptors. Such a method has particular utility in the context of NK cells and especially for the in vitro or ex vivo aspects of the invention, where the use of the agent according to the present invention (i.e. the first agent) may advantageously be combined with the selective expansion of educated NK cells, including NK cells of a given (i.e. selected, or desired) KIR specificity—such a specificity may be selected by selecting donor NK cells of a given KIR specificity. This is discussed in more detail below.

In one aspect, the present invention provides the first agent as defined herein for use in up- or down-regulating the activity of a granular immune effector cell in therapy. Alternatively understood, the invention provides a method of treatment comprising administering an agent as defined herein to a subject, wherein in said method of treatment the activity of a granular immune effector cell is up- or down-regulated. The subject for whom the therapy or method of treatment is provided is preferably a subject suffering from a condition which may be improved by the up- or down-regulation of the activity of one or more of their granular immune effector cells. Such treatment or therapy may be curative or palliative. It is not, however, a requirement that a cure of the condition is achieved; the term “treatment” includes any improvement in the condition or in the clinical status of the subject, which includes any improvement in any symptom or clinical parameter, including for example prolonged survival.

The subject to be treated using the methods of the present invention, and for whom the agent for use in therapy is provided, may be any species of mammal. Thus the subject may be any human or non-human mammalian subject. For instance, the subject may be any species of domestic pet, such as a mouse, rat, gerbil, rabbit, guinea pig, hamster, cat or dog, or livestock, such as a goat, sheep, pig, cow or horse. In a further preferred embodiment of the invention the subject may be a primate, such as a monkey, gibbon, gorilla, orang-utang, chimpanzee or bonobo. Thus, as well as domestic or livestock animals, the subject may be a zoo, laboratory or sport animal (e.g. a racing horse or dog etc.) However, in a preferred embodiment of the invention the subject is a human.

When the agent of the present invention is administered to a subject directly, to perform an in vivo method of the invention, or when the agent is provided for use in therapy, the agent may be administered by any suitable route, e.g. intravenously, intramuscularly, orally, topically, or otherwise locally (e.g. direct infusion), via inhalation etc. The agent may be administered in active form, or it may be administered as a prodrug. The agent may be administered in a form in which the active compound is attached to a targeting molecule, e.g. an antibody, in order to direct the active compound to its target. The agent may be administered using a delivery vehicle, such as lipid particles (microsomes), exosomes or membrane particles.

The agent of the present invention may be provided in the form of a pharmaceutical composition for use in up- or down-regulating the activity of a granular immune effector cell in therapy. Such a pharmaceutical composition may also comprise a ligand, agonist, or antagonist of an inhibitory receptor expressed by the target cell and/or an activator or inhibitor of a signalling pathway downstream of said inhibitory receptor. In addition to said agent and said optional ligand, agonist, antagonist, activator or inhibitor, the pharmaceutical composition also comprises one or more pharmaceutically acceptable diluents, carriers or excipients.

Such compositions may comprise buffers such as neutral buffered saline, phosphate buffered saline and the like; carbohydrates such as glucose, mannose, sucrose, dextrans or mannitol; proteins; polypeptides or amino acids such as glycine; antioxidants; chelating agents such as EDTA or glutathione; adjuvants (e.g. aluminium hydroxide); and preservatives. Compositions of the present invention are preferably formulated for intravenous or oral administration though may be formulated for any other suitable administration as appropriate.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as a solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as sodium chloride or dextrose. The parenteral preparation may be enclosed in ampoules, disposable syringes or multiple dose vials made of glass or plastic. An injectable pharmaceutical composition is preferably sterile.

In some embodiments, oral compositions may be preferred, e.g. tablets, capsules or oral liquid compositions etc.

Pharmaceutical compositions of the present invention may be administered in a manner appropriate to the disease to be treated. The quantity and frequency of administration will be determined by such factors as the condition of the patient, and the type and severity of the patient's disease, and may be determined by the skilled practitioner, as is well known in the art, although appropriate dosages may also be determined by clinical trials.

Also provided by the invention is a product or kit comprising a first agent which modulates the size, content and/or number of secretory lysosomes in a granular immune effector cell, as defined herein, a second agent selected from a ligand, agonist or antagonist of an inhibitory receptor expressed by the granular immune effector cell or an activator or inhibitor of a signalling pathway downstream of said inhibitory receptor, as defined herein. For the medical uses of the invention the product or kit may be provided as a combined preparation for simultaneous, separate or sequential use in up- or down-regulating the activity of granular immune effector cells in therapy.

As discussed above, the invention also provides a method of preparing a granular immune effector cell for adoptive cell therapy, comprising up-regulating the activity of the granular immune effector cell by contacting said cell ex vivo with an agent as defined herein, optionally in combination with a ligand or agonist of an inhibitory receptor expressed by said cell and/or an activator of a signalling pathway downstream of said inhibitory receptor as defined herein.

The invention also provides an in vitro or ex vivo method of up-regulating the activity of a granular immune effector cell, comprising contacting said cell with an agent which modulates the size, content and/or number of secretory lysosomes in said cell, or otherwise modulates the signalling capacity of the secretory lysosomes in said cell, as defined herein, in combination with a ligand or agonist of an inhibitory receptor expressed by said cell and/or an activator of a signalling pathway downstream of said inhibitory receptor, as defined herein.

Such methods involve contacting the cell with the agent, or more particularly incubating the cells in the presence of the agent(s). Granular immune effector cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumours. In certain embodiments, the cells can be obtained from a unit of blood collected from the subject using any number of techniques known to the skilled person, such as FICOLL™ separation. In one embodiment, cells from the circulating blood of a donor (which may be the recipient subject) are obtained by apheresis. The apheresis product typically contains lymphocytes, including T-cells, monocytes, granulocyte, B-cells, NK cells and other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and/or magnesium or may lack many if not all divalent cations. As would be appreciated by those of ordinary skill in the art, a washing step may be accomplished by methods known to those in the art, such as by using a semi-automated flow-through centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In certain embodiments, immune effector cells are isolated from PBMCs by lysing the red blood cells and depleting the monocytes, for example, by centrifugation through a PERCOLL™ gradient. A specific subpopulation of a desired cell type e.g. NK cells or particular T-cells can be further isolated by positive or negative selection techniques. Thus, the cells may be provided as part of a leukocyte-containing cell preparation, e.g. PBMCs or another blood fraction, or the desired cell type (e.g. NK cells, cytotoxic or helper T-cells, or Tregs etc.) may be selectively isolated prior to the contacting step, using techniques known in the art. For example the desired cell type may be positively and/or negatively selected using cell markers, and/or by cell sorting techniques etc., for example, via negative and/or positive magnetic immuno-adherence or flow cytometry that uses a cocktail of antibodies directed to cell surface markers present on the cells to be positively or negatively selected. CD8⁺ cytotoxic T cells can be obtained by using standard methods. In some embodiments, CD8⁺ cells are further sorted into naive, central memory, and effector cells by identifying cell surface antigens that are associated with each of those types of CD8⁺ cells. In embodiments, memory T-cells are present in both CD62L⁺ and CD62L⁻ subsets of CD8⁺ peripheral blood lymphocytes. PBMC are sorted into CD62L⁻CD8⁺ and CD62L⁺ CD8⁺ fractions after staining with anti-CD8 and anti-CD62L antibodies. In some embodiments, the expression of phenotypic markers of central memory T-cells (T_{CM}) include CD45RO, CD62L, CCR7, CD28, CD3, and CD127 and are negative for granzyme B. In some embodiments, T_{CM} are CD45RO⁺, CD62L⁺, CD8⁺ T-cells. In some embodiments, effector T-cells are negative for CD62L, CCR7, CD28, and CD127, and positive for granzyme B and perforin. In some embodiments, naive CD8⁺ T lymphocytes are characterized by the expression of phenotypic markers of naive T-cells including CD62L, CCR7, CD28, CD3, CD127, and CD45RA. Alternatively, the desired cell type may be selectively isolated after the contacting step.

Conditions for the contacting step will generally be conditions which are well known in the art for promoting or enabling cell growth (e.g. cell proliferation or expansion), or at least maintenance of cells. Appropriate conditions may thus be determined according to techniques well known in the art. Depending on the cell type, the contacting (incubation) conditions may include, for example, stimulating the cells may be stimulated with one or more of the following cytokines: Flt-3 ligand (FL), stem cell factor (SF), megakaryocyte growth and differentiation factor (TPO), IL-3 and IL-6 according to the methods known in the art.

If the agent used is cell impermeable (e.g. a non-cell-permeable PIKfyve inhibitor), the agent may be introduced to the cell using photochemical internalisation (PCI). In PCI, the agent is co-administered to the cell(s) with a photosensitiser (e.g. TPCS2a). Uptake of the agent into the cell may then be initiated using photoinduction. Other methods for introducing compounds into cells are also known in the art, including cell-penetrating peptides etc.

The cells may also be manipulated or modified in other ways during the ex vivo/in vitro culturing step. For example specific desired sub-sets of cells may be expanded, for example by the use of conditions which selectively expand a desired subset, or which promote or support expansion of a selected or desired subset. Thus, for example NK cells may be selected, or selectively expanded, which express self-specific KIR (i.e. NK cells which are self-specific, or educated). More particularly, self-specific NK cells are expanded which have a given KIR specificity. A method for this is described in detail below. In another example, since the lysosomal compartment is strongly connected to cell survival through autophagy and the TFEB/mTOR axis, manipulation of these pathways might also be used (alternatively or additionally) as a means selectively to expand specific subsets of cells, particularly to expand educated (self-KIR expressing) NK cells.

As previously mentioned, an agent of the invention used in the ex vivo or in vitro methods described herein may target the granular immune effector cell at one of two stages: at the priming stage, by affecting the programming that happens during effector development, or during cellular proliferation, by manipulation of cell to cell interactions.

In the in vitro and ex vivo methods of the invention, the granular immune effector cell whose activity is up-regulated may be a primary immune effector cell, i.e. an immune effector cell isolated from a donor or patient. Alternatively, an immune effector cell known in the art that has previously been isolated and cultured may be used. Thus a known cell line may be used, e.g. suitable NK cells include (but are by no means limited to), the NK-92, NK-YS, NK-YT, MOTN-1, NKL, KHYG-1, HANK-1, and NKG cell lines.

In the case that the method of the invention is used to prepare ex vivo immune effector cells for use in adoptive cell therapy, the cells may be autologous, or they may be donor cells, which may be MHC-matched or mismatched to the subject to be treated by the adoptive cell therapy (i.e. the recipient), according to the precise nature of the therapy and the cells used. The donor cells may thus be allogeneic, syngeneic or xenogeneic. Thus, if the immune cell is a T-cell it is preferred that the T-cell is autologous, i.e. it is obtained from the same individual it is to be administered to. If not autologous, the T-cell may be matched to the recipient subject. On the other hand, if the immune cell is an NK cell it is preferred that it is non-autologous, or mismatched (which includes partial mismatch). Where the immune effector cell is a non-autologous cell for therapeutic use (i.e. is a donor cell) it is preferred that it is non-immunogenic, such that it does not, when administered to a subject, generate an immune response which affects, interferes with, or prevents the use of the cells in therapy. Immune effector cells may be naturally non-immunogenic, but NK cells or other immune effector cells may be modified to be non-immunogenic. Naturally non-immunogenic NK cells will not express MHC molecules or only weakly express MHC molecules, or may express non-functional MHC molecules which do not stimulate an immunological response. Immune effector cells which would be immunogenic may be modified to eliminate expression of their MHC molecules, or to only weakly express MHC molecules at their surface. Alternatively, such cells may be modified to express a non-functional MHC molecule.

Any means by which the expression of a functional MHC molecule is disrupted is encompassed. Hence, this may include knocking out or knocking down a molecule of the MHC complex, and/or it may include a modification which prevents appropriate transport to and/or correct expression of an MHC molecule, or of the whole complex, at the cell surface.

In particular, the expression of one or more functional MHC class-I proteins at the surface of a cell of the invention may be disrupted. In one embodiment the cells may be human cells which are HLA-negative and accordingly cells in which the expression of one or more HLA molecules is disrupted (e.g. knocked out), e.g. molecules of the HLA MHC class I complex.

In a preferred embodiment, disruption of MHC class-I may be performed by knocking out the gene encoding β₂-microglobulin, a component of the mature MHC class-I complex. Expression of β₂m may be eliminated through targeted disruption of the β₂-microglobulin gene (β₂m), for instance by site-directed mutagenesis of the β₂m promoter (to inactivate the promoter), or within the gene encoding the β₂m protein to introduce an inactivating mutation that prevents expression of the β₂m protein, e.g. a frame-shift mutation or premature ‘STOP’ codon within the gene. Alternatively, site-directed mutagenesis may be used to generate non-functional β₂m protein that is not capable of forming an active MHC protein at the cell surface. In this manner the β₂m protein or MHC may be retained intracellularly, or may be present but non-functional at the cell surface.

Immune effector cells may alternatively be irradiated prior to being administered to a subject. Without wishing to be bound by theory, it is thought that the irradiation of cells results in the cells only being transiently present in a subject, thus reducing the time available for a subject's immune system to mount an immunological response against the cells. Whilst such cells may express a functional MHC molecule at their cell surface, they may also be considered to be non-immunogenic. Radiation may be from any source of α, β or γ radiation, or may be X-ray radiation or ultraviolet light. A radiation dose of 5-10 Gy may be sufficient to abrogate proliferation, however other suitable radiation doses may be 1-10, 2-10, 3-10, 4-10, 6-10, 7-10, 8-10 or 9-10 Gy, or higher doses such as 11, 12, 13, 14, 15 or 20 Gy. Alternatively, the cells may be modified to express a ‘suicide gene’, which allows the cells to be inducibly killed or prevented from replicating in response to an external stimulus.

Thus, an immune effector cell used in the ex vivo or in vitro methods of the invention may be modified to be non-immunogenic by reducing its ability, or capacity, to proliferate, that is by reducing its proliferative capacity.

In some embodiments of the invention, the granular immune effector cell is derived from an induced pluripotent stem cell (iPSC). iPSCs are pluripotent stem cells which are generated directly from adult cells, by reprogramming the cells to become pluripotent, i.e. able to differentiate into any type of cell. As above, if the granular immune effector cell is to be used in adoptive cell therapy, the iPSCs may be autologous or non-autologous.

In certain embodiments, the granular immune effector of cell whose activity is up-regulated in the in vitro or ex vivo methods of the invention is an NK cell. In such embodiments, as mentioned above, the NK cell may be expanded by stimulating one or both of the paired receptors CD94/NKG2C and CD94/NKG2A. By expanding the NK cells in such a manner, a population of cells with the phenotype NKG2C⁺/NKG2A⁻ is obtained. NKG2C is an activatory NK cell receptor specific for HLA-E, a class I MHC protein whose expression is associated with tumour cells, and thus its expression on the expanded cells is beneficial to enhance their activity. NKG2A is an inhibitory receptor, also specific for HLA-E. Lack of expression of the NKG2A inhibitory receptor is also beneficial in enhancing activity of the cells. Expansion of NK cells with stimulation of one or both of the paired receptors CD94/NKG2C and CD94/NKG2A thus yields a population of cells with the phenotype NKG2C⁺/NKG2A⁻. Such a population is useful in the treatment of cancer as it is particularly active against cells expressing the HLA-E protein. This technique is further discussed in WO 2014/037422, in which the inventors showed that the selective expansion of educated cells by stimulation of the paired receptors CD94/NKG2C (activating receptor) and CD94/NKG2A (inhibitory receptor) (which as described above produces cells which are NKG2C-positive and NKG2A-negative) may increase the survival and/or proliferation of the cells.

As noted above, by selecting donor cells of a given KIR specificity, selected with respect to the MHC class I type of the subject, and optionally with respect to the condition to be treated, a population of cells may be generated for adoptive cell therapy which is personalised to the subject. The method selectively expands the NK cells of the selected KIR specificity. More particularly, the expanded KIR cells express self-KIR receptors of a given (selected) KIR specificity. The donor NK cells may be selected to have an at least partial mismatch at HLA class I between the donor NK cells and the recipient subject, or more particularly the target cells in the subject for the adoptive cell therapy. However, where the target cells for the therapy lack or are deficient in expression of MHC class I, the donor cells may be matched to the recipient subject.

Briefly, the method involves contacting the isolated donor cells (which may be selectively isolated NK cells or a preparation of cells which contains NK cells, e.g. a preparation of leukocytes such as PBMC etc.) with the stimulator under conditions which promote or enable cell expansion. Such conditions are well-known in the art, as discussed above. As is well-known in the art, additional signals for NK cell expansion may be provided through a multitude of receptors, including but not limited to CD16, NKG2D, NKp46, CD2, 2B4, DNAM-1 and CD137, or a combination thereof. The stimuli for such receptors may be provided by appropriate feeder cells or by complexes of the stimulatory molecules or receptor ligands e.g. on beads. As noted above, techniques for this are well established. Cytokines may additionally be added, including but not limited to IL-5, IL-15, IL-12, IL-18, IL-2, IL-7, IL-21 or IFN-alpha or a combination with one or more of these cytokines. These cytokines or other agents can be used to stimulate proliferation and/or promote apoptosis. In some embodiments the cells are cultured with IL-15.

The stimulator of NKG2C and/or NKG2A may be any ligand, natural or synthetic, that can bind to and stimulate one or both of these receptors or their constituent monomers. In other words, the stimulator is an agonist of the NKG2C and/or NKG2A receptors. Thus, the stimulator may be a ligand or agonist for CD94, or for NKG2C and/or NKG2A. As indicated above, the stimulator is preferably the natural ligand HLA-E. HLA-E needs to be provided in a form in which it is able to stimulate the cells, and again this is well understood in the art. Thus the HLA-E may be provided in complex with leader sequence peptides. For example leader sequence peptides can be derived from HLA-A, HLA-B, HLA-C or HLA-G molecules. It is known in the art how to insert a sequence encoding the leader peptide into genetic constructs expressing HLA-E so that the HLA-E is expressed in a complex form capable of binding to and stimulating the NKG2C/NKG2A receptors. Cell lines expressing such constructs are known and available, for example the cell line 721.221.AEH that expresses HLA-E in complex with the leader sequence of HLA-A. Such cell lines may be used as feeder cells to provide HLA-E to the donor NK cells.

In other embodiments, HLA-E multimers, e.g. tetramers or pentamers, may be used, together with any relevant peptide that provides a signal to CD92/NKG2A and/or CD92/NKG2C. Such multimers may be coated onto an appropriate solid support, e.g. beads, membrane particles or plastic supports or vessels e.g. plates or bags, to provide the desired combined stimulation/inhibition effects, as described above. In yet other embodiments the stimulator may be an antibody which binds to one or more of CD94, NKG2C or NKG2A. It will be understood that this will be an agonistic antibody, namely an antibody which is capable of stimulating the receptor. Combinations of such antibodies may be used, for example an antibody which binds to NKG2C may be used together with an antibody that binds to NKG2A. Preferably the antibody is anti-NKG2C, or anti-NKG2C together with anti-NKG2A. The antibody may be a bi-specific antibody. In particular, the antibody may be a bi-specific killer cell engager (BiKE), for instance a BiKE with dual specificity for NKG2A and NKG2C, or dual specificity for CD94 and one of NKG2A or NKG2C. The antibody may alternatively be a tri-specific killer cell engager (TriKE), for instance a TriKE with tri-specificity for NKG2A, NKG2C and CD94.

Thus the step of expanding the NK cells may be achieved by methods including but not limited to (a) using a feeder cell line engineered by different means to express HLA-E (including but not limited to 721.221.HLA-E or K562-HLA-E, or variants of these cell lines modified to express (or overexpress) agonists stimulating for example IL-15 and/or IL-21 receptors, or ligands for activating receptors such as PVR, CD48 or ICAM-1); or (b) plates or bags coated in soluble HLA-E complexes alone or in combination with cytokine receptor complexes; or (c) plates or bags coated with anti-NKG2C mAbs and/or anti-CD94 and/or anti-NKG2C and/or anti-NKG2A mAbs; or (d) beads coated with soluble HLA-E complexes and/or anti-CD94 and/or anti-NKG2C and/or anti-NKG2A mAbs. The HLA-E construct used in the embodiments described in (a) and (b) uses the generic molecule or a modified construct with improved stability and/or binding to the CD94/NKG2A/C molecules based on changes in the leader sequence peptides. These include but are not limited to HLA molecules coupled to the HLA-A and HLA-G leader and peptide modifications thereof. Leukocytes (PBMC) or isolated NK cells can be cultured under conditions known in the art

A cell or population of cells produced by the in vitro or ex vivo methods of the invention is also provided. Such cells or cell populations may be further modified to express one or more antigen receptors that would not otherwise be expressed by the cell or cell population. Examples of such antigen receptors include chimeric antigen receptors (CARs) and T-cell receptors (TCRs). T-cells can easily be modified to express functioning CARs and non-native TCRs. The use of CARs in NK cells has long been described. The cells of the invention may be modified to express an antigen receptor against any desired target antigen, in particular a cancer-specific antigen. The expression of a CAR or TCR to a selected tumour target antigen may be used to expand the range of cancers that may be targeted by the adoptive cell therapy. Thus, cancers may be included which are not only those naturally targeted by NK cells for example. Genetic modification of the cells to introduce nucleic acid molecules encoding a desired antigen receptor may be performed before, during or after a culture step, or more particularly the step of contacting the cells with the agent.

Alternatively, or additionally, the cell or population of cells produced by the in vitro or ex vivo methods of the invention may be modified to express a chemokine receptor. In particular, the chemokine receptor may be a cytokine receptor, in particular TNF receptor, a TGFβ receptor or an interleukin receptor.

The cell(s) or population of cells provided by the in vitro or ex vivo methods of the invention may be provided in the form of a pharmaceutical composition. Such a composition comprises a cell or population of cells produced by an in vitro or ex vivo method of the invention together with one or more pharmaceutically acceptable diluents, carriers or excipients. Such diluents, carriers or excipients are defined above.

The cell or population of cells produced by the in vitro or ex vivo methods of the invention, or a pharmaceutical composition comprising such a cell or cell population, may be used in therapy or a method of treatment. Such therapy or treatment comprises administering the cell, cell population or pharmaceutical composition to a subject. The subject is as defined above. “Treatment” as used herein is also defined above. The therapy is preferably adoptive cell therapy. The cell, cell population or pharmaceutical composition may be administered to the subject by any appropriate method, but is preferably administered intravenously. Appropriate dosages, frequencies of administration and suchlike can be calculated by the skilled person based on experience and clinical trials.

The cell or population of cells may be administered to the subject in combination with a reagent which counters cell adhesion, in order to promote wide circulation of the cells throughout the subject's body. Alternatively a pharmaceutical composition of the invention which comprises cells or a population of cells may further comprise such an agent. Examples of such agents include BIRT377.

The therapy and methods of treatment of this invention can be used in the treatment of any condition which may benefit from the up-regulation of the activity of an immune effector cell. In particular, the invention may be particularly useful in the treatment of cancer, an inflammatory disorder (particularly an autoimmune disorder), an immunodeficiency, a lysosomal disorder or an infection.

Cancer is defined broadly herein to include any neoplastic condition, whether malignant, pre-malignant or non-malignant. Generally, however, it may be a malignant condition. Both solid and non-solid tumours are included and the term “cancer cell” may be taken as synonymous with “tumour cell”.

Any type of cancer is encompassed, including both solid and haematopoietic cancers. Representative cancers include Acute Lymphoblastic Leukaemia (ALL), Acute Myeloid Leukaemia (AML), Adrenocortical Carcinoma, AIDS-Related Cancer (e.g. Kaposi Sarcoma and Lymphoma), Anal Cancer, Appendix Cancer, Astrocytomas, Atypical Teratoid/Rhabdoid Tumour, Basal Cell Carcinoma, Bile Duct Cancer, Extrahepatic Bladder Cancer, Bone Cancer (e.g. Ewing Sarcoma, Osteosarcoma and Malignant Fibrous Histiocytoma), Brain Stem Glioma, Brain Cancer, Breast Cancer, Bronchial Tumours, Burkitt Lymphoma, Carcinoid Tumour, Cardiac (Heart) Tumours, Cancer of the Central Nervous System (including Atypical Teratoid/Rhabdoid Tumour, Embryonal Tumours, Germ Cell Tumour, Lymphoma), Cervical Cancer, Chordoma, Chronic Lymphocytic Leukemia (CLL), Chronic Myelogenous Leukaemia (CML), Chronic Myeloproliferative Disorder, Colon Cancer, Colorectal Cancer, Craniopharyngioma, Cutaneous T-Cell Lymphoma, Bile Duct Cancer, Extrahepatic Ductal Carcinoma In Situ (DCIS), Embryonal Tumours, Endometrial Cancer, Ependymoma, Esophageal Cancer, Esthesioneuroblastoma, Ewing Sarcoma, Extracranial Germ Cell Tumour, Extragonadal Germ Cell Tumour, Extrahepatic Bile Duct Cancer, Eye Cancer (including Intraocular Melanoma and Retinoblastoma), Fibrous Histiocytoma of Bone, Gallbladder Cancer, Gastric (Stomach) Cancer, Gastrointestinal Carcinoid Tumour, Gastrointestinal Stromal Tumours (GIST), Germ Cell Tumor, Gestational Trophoblastic Disease, Glioma, Hairy Cell Leukaemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumours, Pancreatic Neuroendocrine Tumours, Kaposi Sarcoma, Kidney Cancer (including Renal Cell and Wilms Tumour), Langerhans Cell Histiocytosis, Laryngeal Cancer, Leukaemia (including Acute Lymphoblastic (ALL), Acute Myeloid (AML), Chronic Lymphocytic (CLL), Chronic Myelogenous (CML), Lip and Oral Cavity Cancer, Liver Cancer (Primary), Lobular Carcinoma In Situ (LCIS), Lung Cancer, Lymphoma, Macroglobulinemia, Waldenström, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Childhood, Multiple Myeloma/Plasma Cell Neoplasm, Mycosis Fungoides, Myelodysplastic Syndromes, Myelodysplastic/Myeloproliferative Neoplasms, Multiple Myeloma, Myeloproliferative Disorders, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumours (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sézary Syndrome, Skin Cancer, Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Urethral Cancer, Uterine Cancer, Endometrial, Uterine Sarcoma, Vaginal Cancer, Vulvar Cancer, Waldenström Macroglobulinemia, and Wilms Tumour.

Particular mention may be made of haemopoietic cancers (e.g. any type of leukaemia, including childhood or adult, chronic or acute, myeloid or lymphoid, e.g. AML, CML, ALL, CLL, any type of lymphoma, malignant melanoma, myelodysplastic syndrome), glioblastoma, melanoma, prostate cancer, ovarian cancer, colorectal cancer, renal cell cancer, breast cancer and pancreatic cancer, or any subset thereof. In the context of cancer therapy, up-regulation of granular immune effector cell activity would generally be desired, in order to increase immune system activity against the cancer cells. In the context of the methods of adoptive cell therapy of the invention, up-regulated cytotoxic immune cells, in particular CTLs and NK cells, may be used in the treatment of cancer. Up-regulated T-helper cells are also useful in the treatment of cancer.

Other conditions which may be treated or prevented according to the present invention include inflammatory conditions (inflammatory conditions include autoimmune disorders). This includes any inflammatory condition, which term is used broadly herein to include any inflammatory disease or any condition having an inflammatory component, including conditions associated with acute or chronic inflammation. “Chronic inflammation” generally means an inflammation (e.g. an inflammatory condition) that is of persistent or prolonged duration in the body of a subject. Generally speaking this means an inflammatory response or condition of duration of 20, 25 or 30 days or more or 1 month or more, more particular of at least 2 or 3 months. Chronic inflammation leads to a progressive shift in the type of cells present at the site of inflammation. Chronic inflammation may occur as a result of persistent or prolonged injury or infection, prolonged exposure to toxic substances or by autoimmune responses or conditions. Chronic inflammation may be a factor in the development of a number of diseases or disorders, including particularly degenerative diseases, or diseases or conditions associated with loss of youthful function or ageing.

Included under inflammatory conditions are conditions associated with systemic inflammation, that is inflammation which is not confined to a particular tissue or site or location in the body, or alternatively involve local inflammation, including local internal inflammation.

Exemplary inflammatory conditions include inflammatory bowel disease (also classified as an autoimmune condition), osteoarthritis and other forms of arthritis, cancer-associated inflammation, cardiovascular diseases (i.e. CVD which is associated with inflammation or has an inflammatory component), non-alcoholic Steatohepatitis (NASH), and nephritis, as well as any inflammation associated with infection. Effectively, any inflammatory condition may be treated where it is of clinical value to dampen the inflammation. As noted above, the agents used according to the invention may modulate the production of cytokines and thus may be used in the context of any condition involving an undesirable or elevated cytokine profile. The invention may be used to treat any autoimmune disorder, including multiple sclerosis (MS), inflammatory bowel diseases including Crohn's disease and ulcerative colitis, systemic lupus erythematosus (SLE), ankylosing spondylitis, juvenile arthritis, rheumatoid arthritis, spondyloarthritis, psoriasis, systemic sclerosis (scleroderma), type 1 diabetes, polymyalgia rheumatica (PMR) and interferonopathies. Autoimmune diseases as herein defined include conditions or syndromes which cause or result in cytokine storms, including hemophagocytic lymphohistiocytosis (HLH), and familial hemophagocytic lymphohistiocytosis (FHL).

In the context of an autoimmune disorder, down regulation of immune effector cell activity would generally be desired. In the context of the methods of adoptive cell therapy of the invention, inflammatory conditions, including autoimmune disorders, may be treated using up-regulated Treg cells, in order to reduce the activity of CTLs and T-helper cells, thus reducing the undesired immune response. Up-regulated Treg cells may also be used in the treatment of graft-versus-host-disease (GvHD) following allogeneic stem cell transplantation.

A lysosomal disorder is any disorder in which lysosomal function is defective. Lysosomal disorders may be caused by a deficiency or mutation in one or more enzymes required for macromolecule metabolism, including the metabolism of proteins, lipids, glycoproteins and glycosaminoglycans. Other causes include defects in the transport of substrates into and out of lysosomes. The majority of lysosomal disorders are genetic.

It is proposed that by modulating lysosome size, content and/or number according to the invention, lysosomal activity may be improved or restored. Any lysosomal disorder may be treated by the in vivo therapies and methods of treatment of the invention, including Niemann-Pick syndrome (including Types A, B and C), Fabry disease, Farber disease, Schindler disease, Sandhoff disease, LAL deficiency, Sanfilippo syndrome, Sly syndrome, !-cell disease, sialidosis, cystinosis, pycnodysostosis, Morquio syndrome, Hunter syndrome, Tay-Sachs disease, Gaucher's disease, metachromatic leukodystrophy, multiple sulfatase deficiency, galactosialidosis, and Salla disease.

Infections may also be treated by the current invention, by up-regulating the activity of granular immune effector cells against infected cells, that is cells infected with any pathogen. Typically such cells will be virus-infected cells, but they may also be infected with any other pathogenic organism, e.g. any microorganism, for example, bacteria, fungi, mycoplasma, protozoa or prions. In the context of the methods of adoptive cell therapy of the invention, up-regulated CTLs, T-helper cells and NK cells may in particular be used to treat infections.

Alternatively, immune effector cells up-regulated according to the present invention may be used to target cells in the subject which may be apoptotic or pre-apoptotic, or be in a stressed state (i.e. express stress-related markers at their cell surface), or may be a mutant cell, e.g. expressing a particular mutation.

The present invention may be more fully understood from the Examples below and in reference to the drawings, in which:

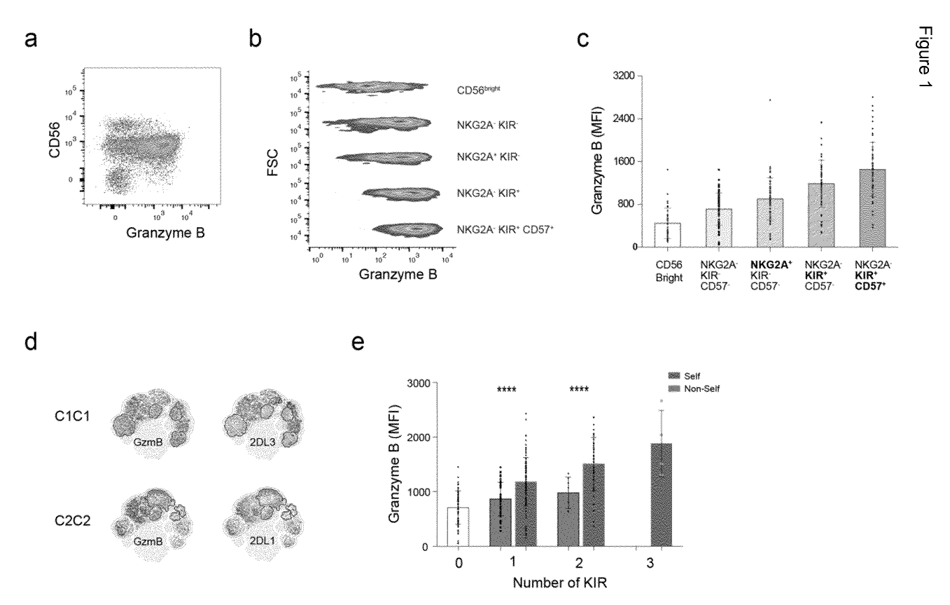

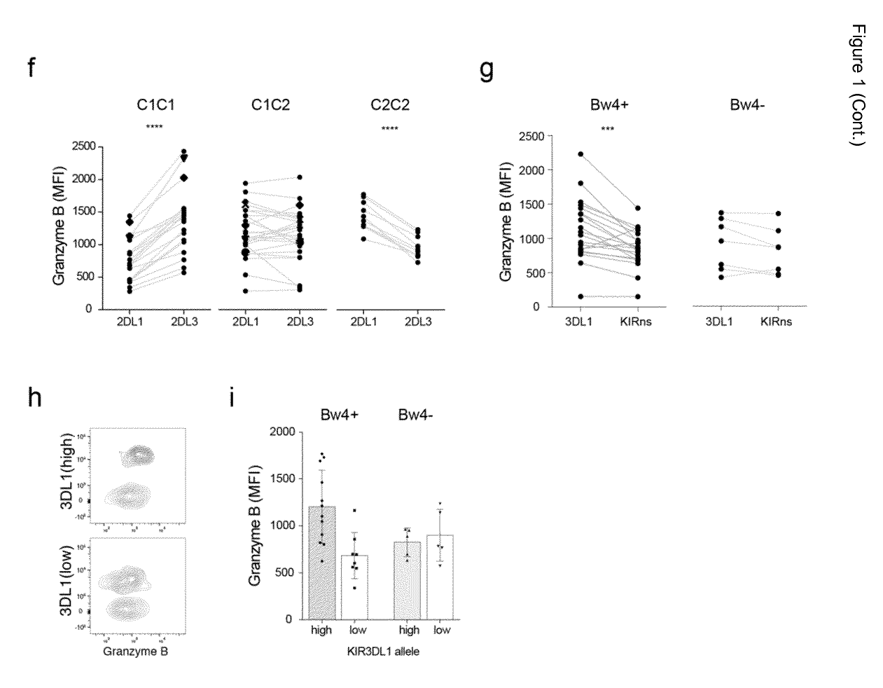

FIG. 1 shows that granular loading of NK cells is determined by NK cell differentiation and inhibitory input through self-specific KIR. A-C show expression of granzyme B in the indicated NK cell subsets. D is an SNE plot showing intensity of granzyme B in clusters defined by 2DL3 and 2DL1 expression in C1/C1 and C2/C2 donors, respectively. E shows expression of granzyme B in subsets of NK cells expressing 0, 1-2 Non-Self or 1-3 Self KIR. F shows expression of granzyme B in 2DL3 and 2DL1 single-positive NK cells from C1/C1, C1/C2 and C2/C2 donors. G shows expression of granzyme B in 3DL1⁺ and KIR⁻ NK cells from Bw4⁺ and Bw4⁻ donors. KIRns=KIR non-self H & I show expression of granzyme B in 3DL1^{high} and 3DL1^{low} NK cells from Bw4⁻ and Bw4⁻ donors.

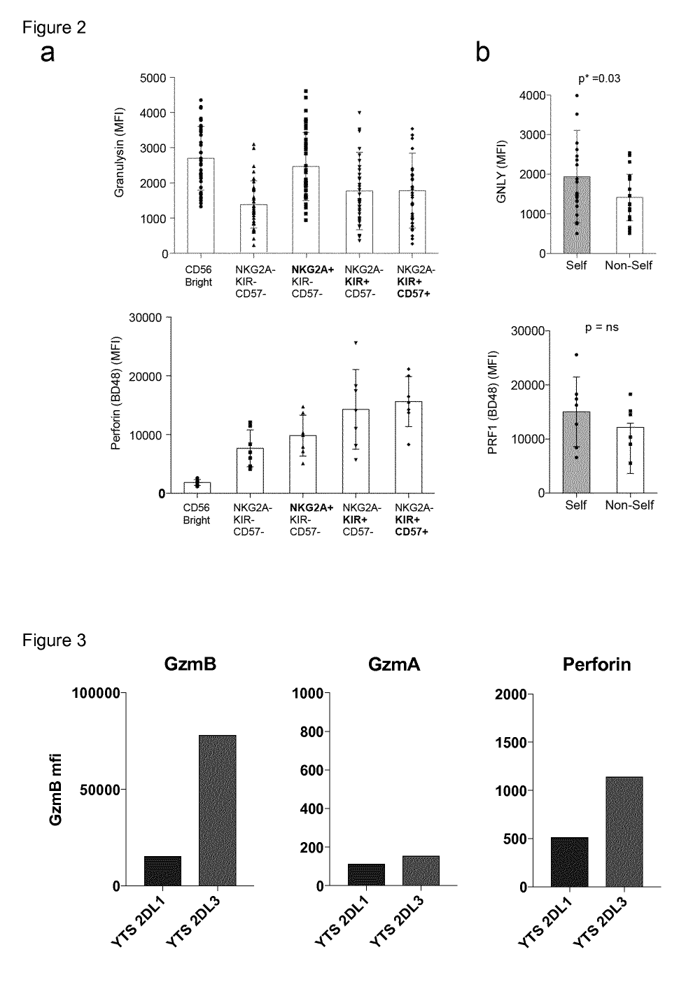

FIG. 2 shows that granular loading is determined by NK cell differentiation and inhibitory input through self-specific KIR. A & B both show expression levels of Granulysin and Perforin in the indicated NK cell subsets. P=ns indicates no statistically significant difference.

FIG. 3 shows that the introduction of a self-KIR receptor (2DL3) into the NK cell line YTS (HLA-C1+) leads to accumulation of granzyme B and perforin, replicating the educated state.

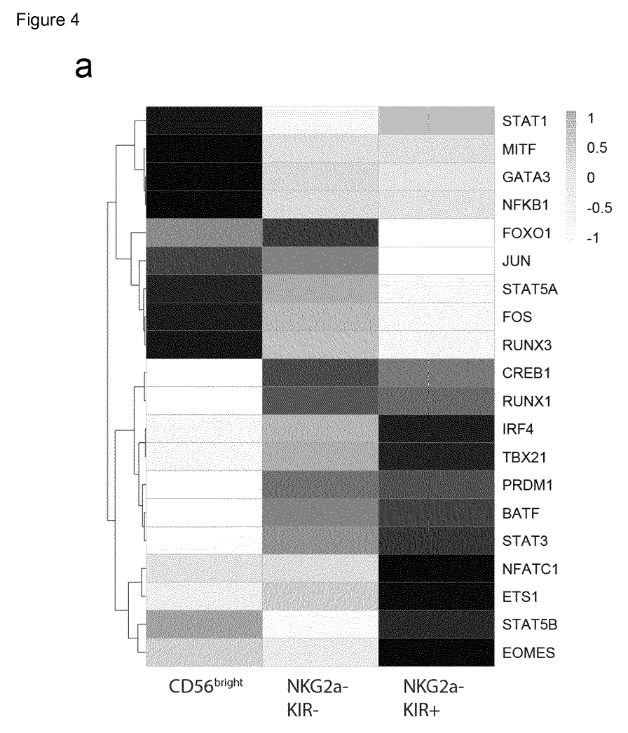

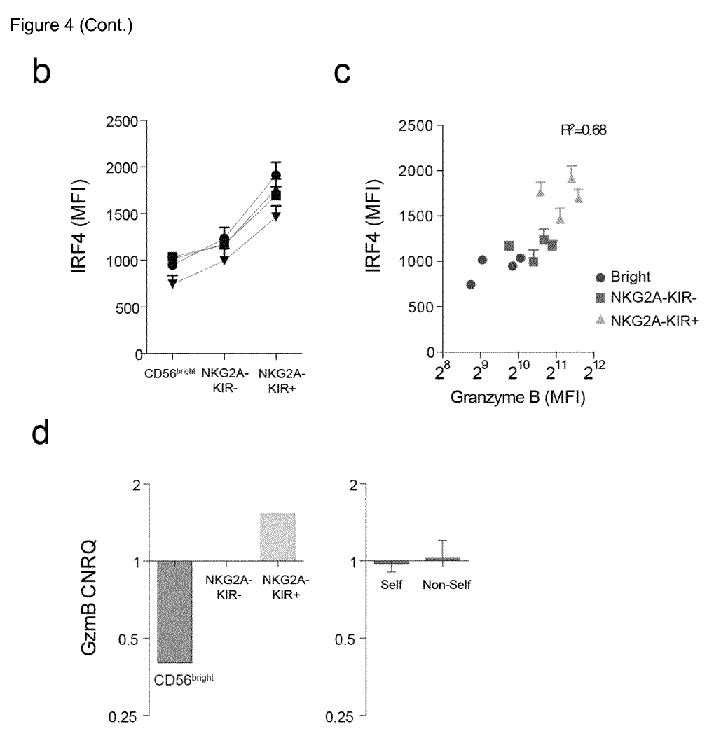

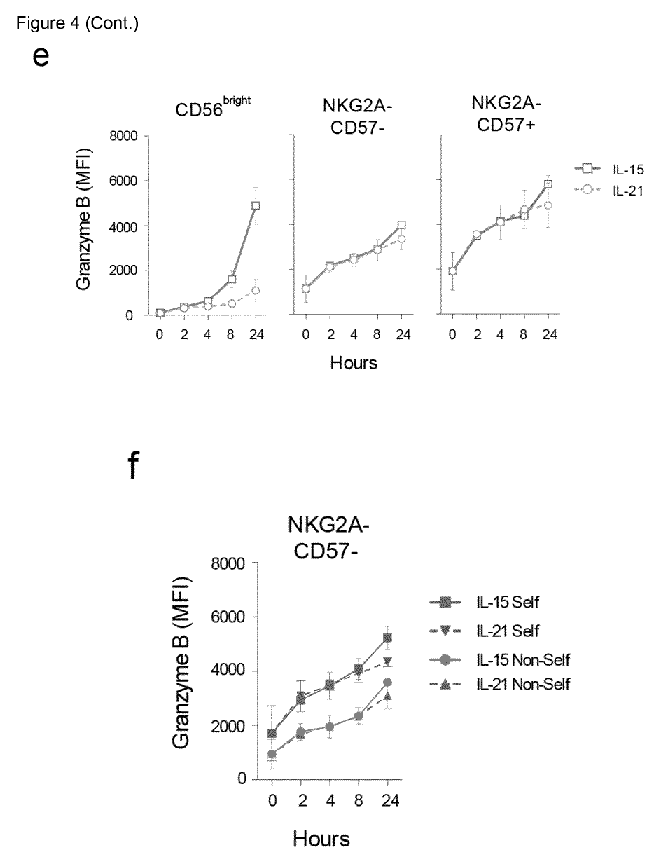

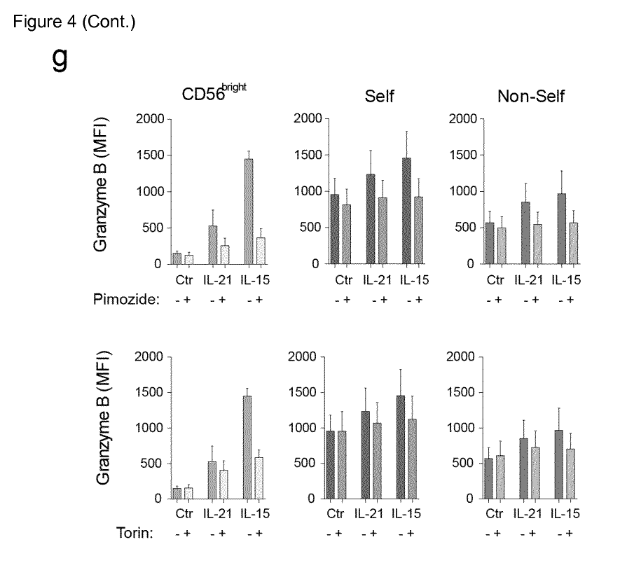

FIG. 4 shows that accumulation of granzyme B in educated NK cells is independent of transcription and translation. A shows mRNA expression levels (measured by RNA Seq) of the indicated genes in CD56^{bright}, NKG2A⁻KIR⁻ and NKG2A⁻KIR⁺ CD56^{dim} NK cell subsets. B shows expression levels of IRF4 protein in the indicated NK cell subsets. C shows a correlation between IRF4 and granzyme B protein expression in discrete subsets of NK cells during differentiation. D shows results of quantitative PCR of granzyme B mRNA in cD56^{bright}, NKG2A⁻KIR⁻ and NKG2A⁻KIR⁺ CD56^{d}″NK cell subsets and in NKG2A⁻KIR⁺ CD56^{dim} NK cells expressing Self and Non-Self KIR. E & F show expression levels of granzyme B in the indicated NK cell subsets following stimulation with IL-15 or IL-21 for the indicated length of time. G shows expression levels of granzyme B after 24 h of stimulation with IL-15 or IL-21 in the presence or absence of the STAT-5 inhibitor Pimozide (top) and the mTOR inhibitor Torin (bottom).

FIG. 5 (in conjunction with FIG. 3) shows transcriptional dissociation of differentiation and education. Results are shown of quantitative PCR of mRNA of 8 markers showing a positive relationship with differentiation. qPCR was performed in CD56^{bright}, NKG2A⁻KIR⁻ and NKG2A⁻KIR⁺ CD56^{dim} NK cell subsets and in NKG2A⁻KIR⁺ CD56^{dim} NK cells expressing Self and Non-Self KIR as shown. For each marker, results from CD56^{bright}, NKG2A⁻KIR⁻ and NKG2A⁻KIR⁺ CD56^{dim} subsets are presented from left to right in the left-hand graphs. Results from NKG2A⁻KIR⁺ CD56^{dim} subsets expressing Self and Non-Self KIR are presented from left to right in the right-hand graphs.

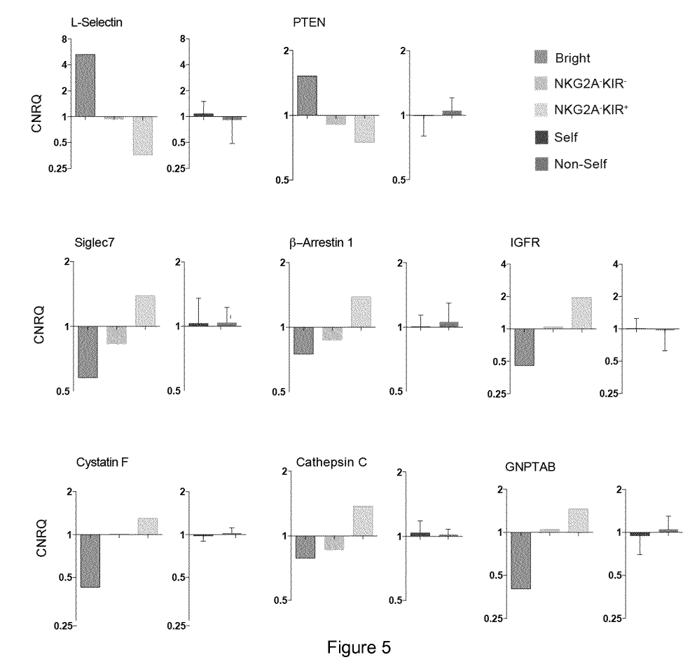

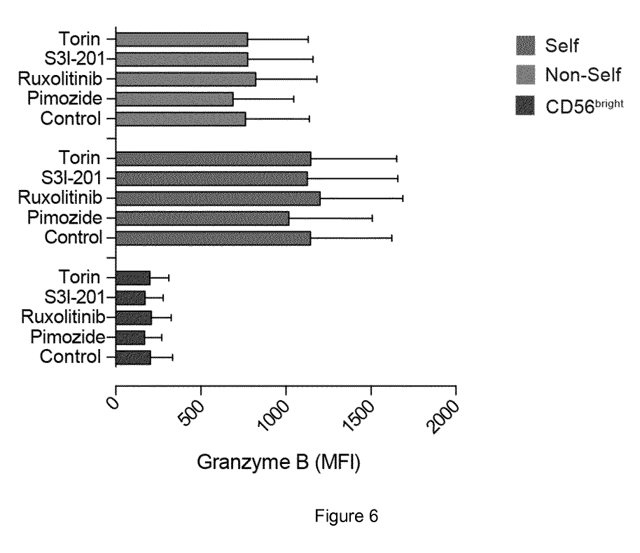

FIG. 6 shows that constitutive granzyme B expression in Self-KIR⁺ and Non-Self KIR⁺ NK cells is independent of baseline activity of mTOR and STAT signalling. Expression levels of granzyme B in CD56^{bright} and NKG2A⁻KIR⁺ CD56^{dim} NK cells expressing Self and Non-Self KIR following treatment with the indicated inhibitors of mTOR (Torin), Stat3 (S3I-201), Janus kinase (Ruxolitinib) and StatS (Pimozide) are shown. Results from the NKG2A⁻ KIR⁺ CD56^{dim} NK cell subset expressing Non-Self KIR are shown at the top; results from the NKG2A⁻KIR⁺ CD56^{dim} NK cell subset expressing Self KIR are shown in the middle; results from the CD56^{bright} NK cell subset are shown at the bottom.

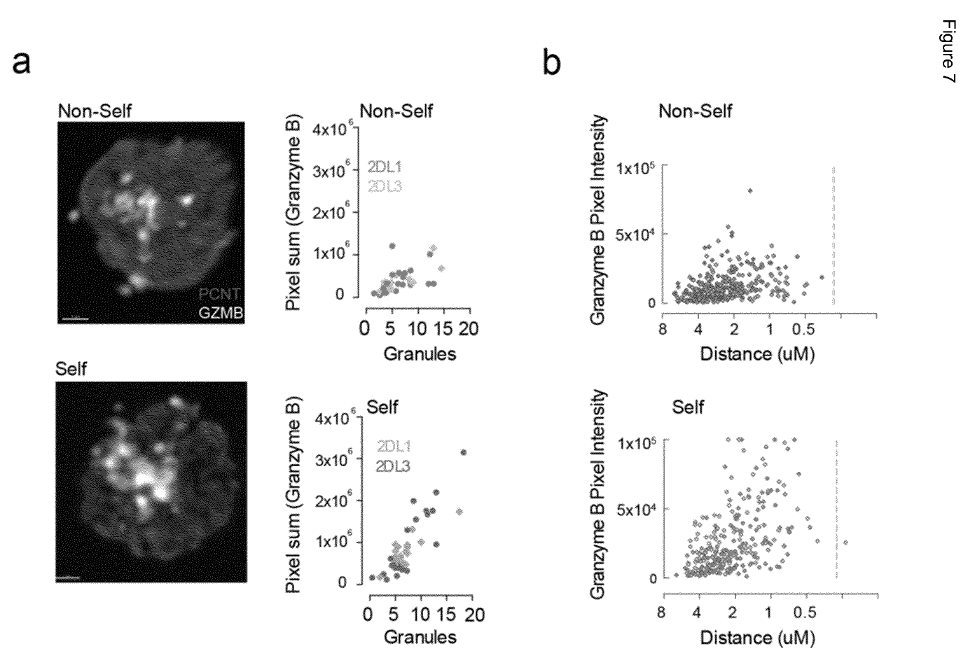

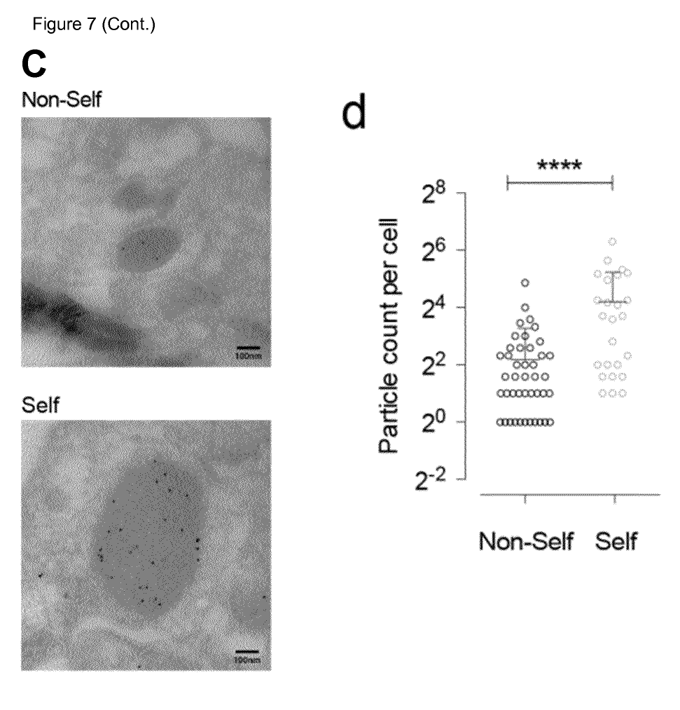

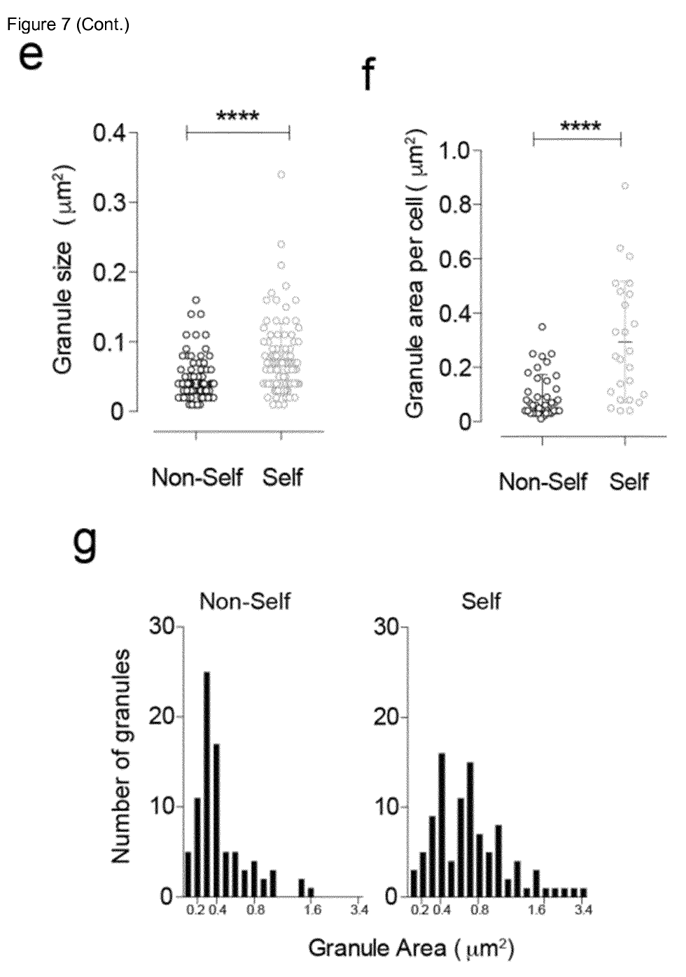

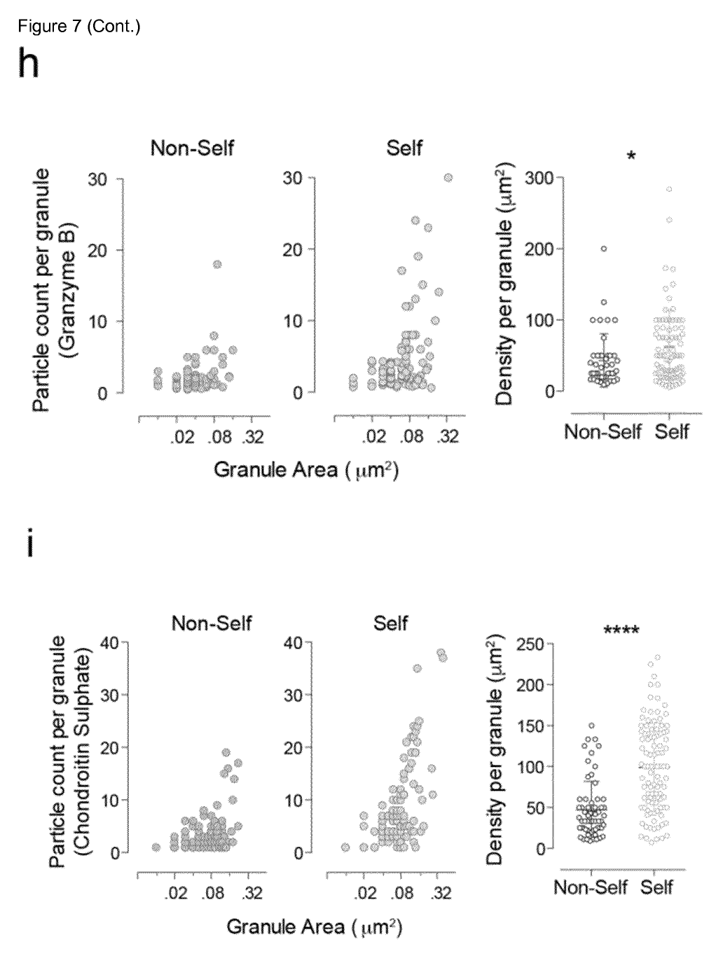

FIG. 7 shows that expression of self-KIR is associated with accumulation of large granzyme B-dense secretory lysosomes. In A a representative confocal microscopy Z-stack is presented, showing Pericentrin (PCNT) and granzyme B staining in sorted CD56^{dim} NKG2A⁻CD57⁻ NK cells expressing Non-Self or Self KIR is shown on the left. The pixel sum of granzyme B staining in cells expressing Non-Self or Self KIR versus the number of granules is presented on the right. Data are aggregated from sorted 2DL1 and 2DL3 single-positive NK cell subsets from C1C1 (n=3 or 5) and C2C2 (n=2 or 5) donors. B shows the relationship between granzyme B expression levels in individual granules in NK cells expressing Non-Self or Self KIR and the distance from the centrosome. In C representative immuno-EM images are presented, showing staining with gold particle-coated anti-granzyme B antibodies of sorted CD56^{dim} NKG2A⁻CD57⁻ NK cells expressing Non-Self or Self KIR. D shows the number of gold particles per cell, E shows the size of the granules in the cells (Non-Self n=83, Self n=98) and F shows the granular area per cell in Non-Self (n=41) and Self KIR+ NK cells (n=25) from n=3 donors. In H and I data is presented showing the number of gold particles per granule following granzyme B staining (H) or Chondroitin Sulphate staining (I) relative to the granular area in sorted Non-Self and Self KIR+ NK cells (left panels) and the relative density of gold particles per granule in granules with area >0.04 μm² (right panels).

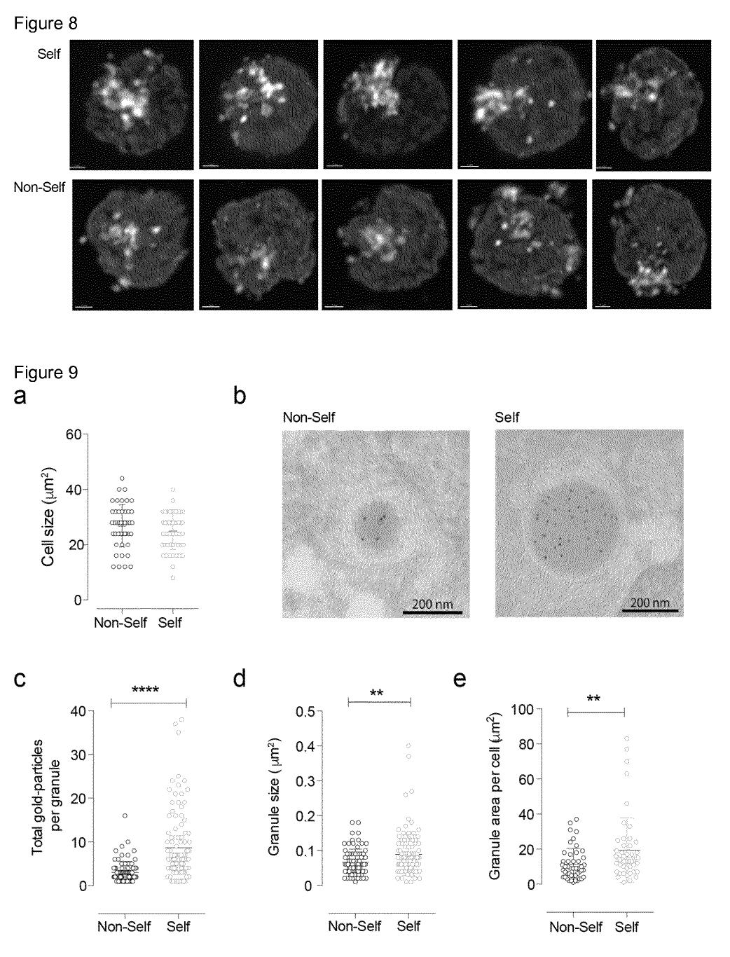

FIG. 8 shows that expression of self-KIR is associated with accumulation of large granzyme B-dense secretory lysosomes. Confocal Z-stacks showing Pericentrin (PCNT) and granzyme B staining in sorted CD56^{dim} NKG2A⁻CD57⁻ NK cells expressing Self (top) or Non-Self KIR (bottom) are presented. A panel of representative phenotypes acquired from one representative donor out of 5 is shown.

FIG. 9 shows increased density of chondroitin sulphate 4 (CS4) within granules from Self-KIR⁺ NK cells. A shows a comparison of cell size between self- (n=50) and non-self-(n=49) specific NK cells. In B a representative immuno-EM image is presented, showing staining with gold particle-coated anti-CS4 antibodies of sorted CD56^{dim} NKG2A⁻ CD57⁻ NK cells expressing Non-Self or Self KIR. C shows the number of gold particles (from CS4 staining) per granule (Non-Self n=83, Self n=109). D shows the granular size, and E the granular area, of Non-Self or Self KIR+ NK cell, as determined by measuring of CS4⁺ granules.

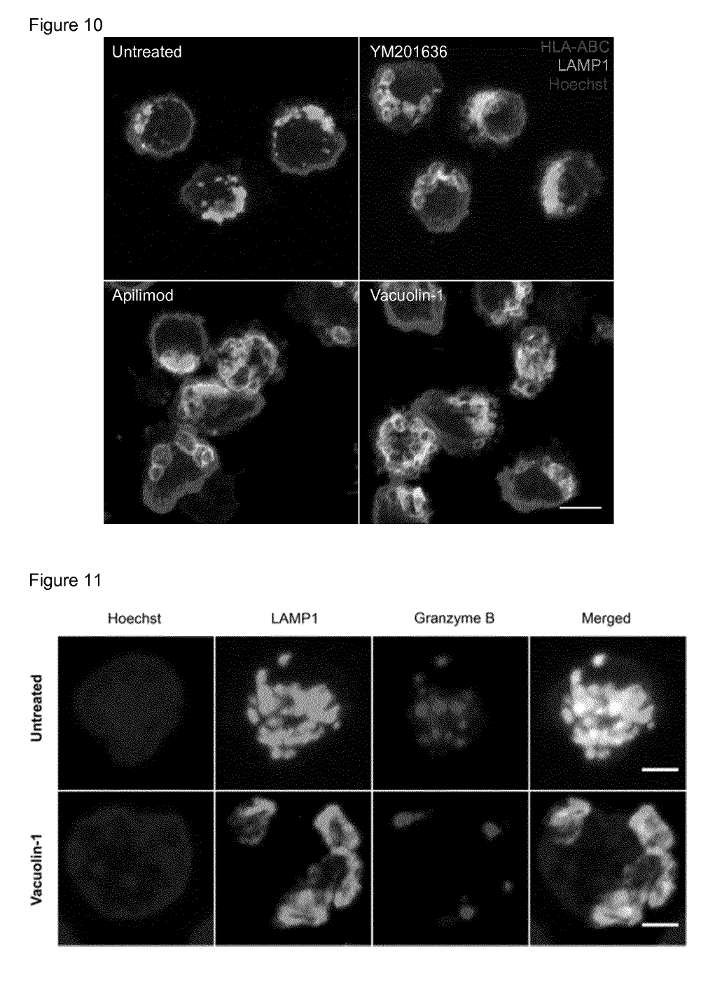

FIG. 10 shows the results of confocal imaging of primary NK cells exposed to various PIKfyve inhibitors. Lysosomal size, as illustrated by LAMP-1 staining, is increased following pharmacological inhibition of PIKfyve.

FIG. 11 demonstrates the clustering of Granzyme B inside the secretory lysosomes of primary NK cells after treatment with vacuolin-1.

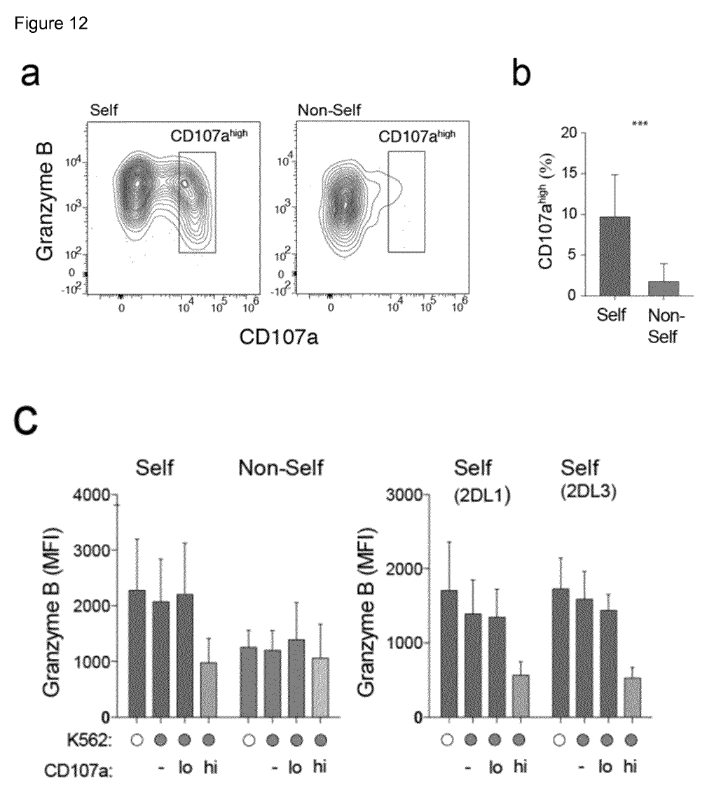

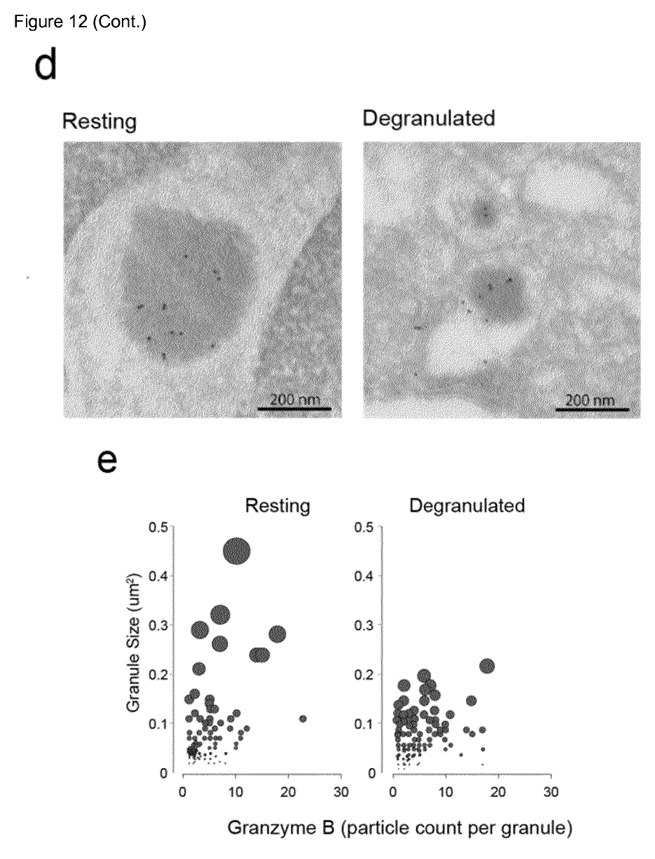

FIG. 12 shows threshold release of large cytotoxic granules upon target cell stimulation. A shows a representative example of granzyme B and CD107a expression in Self KIR⁺ and Non-Self KIR⁺ CD56^{dim} NKG2A⁻CD57⁻ NK cells following stimulation with K562 cells. B presents aggregated data showing the percentage of CD107a^{high} NK cells following stimulation of Self KIR⁺ and Non-Self KIR⁺ CD56^{dim} NKG2A⁻CD57⁻ NK cells with K562 cells (n=5). C shows expression levels of granzyme B in the indicated NK cell subset (all NK cells; CD107a negative (−), low or high) after stimulation with K562 cells. Left graph: C1/C1 donors (n=4), Right graph: C1/C2 donors (n=4). In D a representative Immuno-EM image of resting or sorted CD107a^{High} NK cells is shown. E shows the granular size and granzyme B content as determined by immuno-EM in resting and sorted CD107a^{High} NK cells after stimulation with K562 cells.

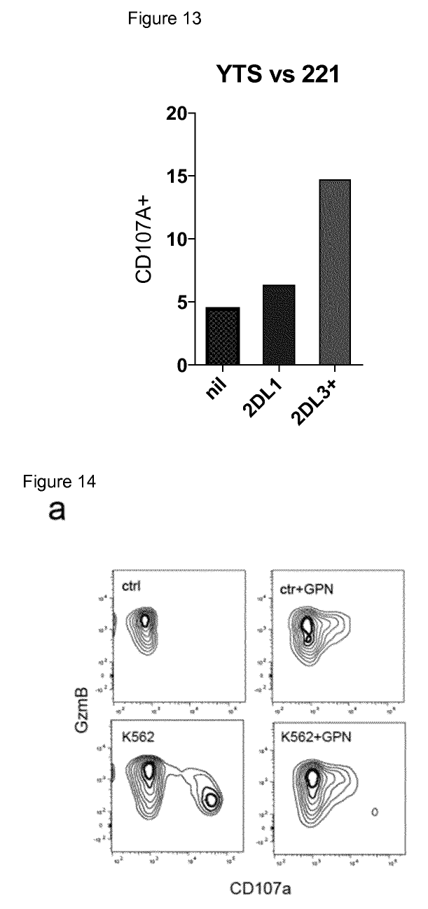

FIG. 13 shows that the introduction of a self-KIR receptor (2DL3) into the NK cell line YTS leads to increased functionality as illustrated by the up-regulation of CD107a in response to 221 target cells.

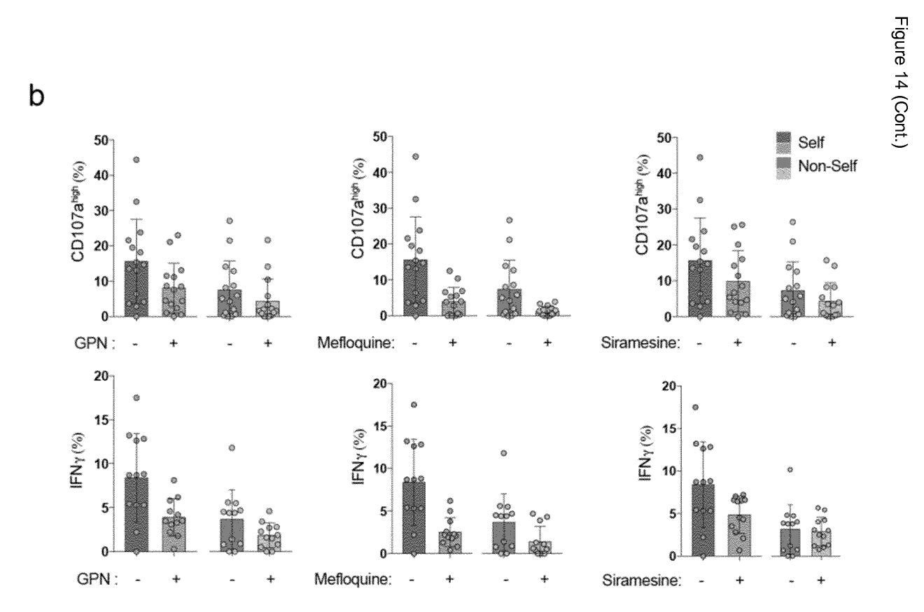

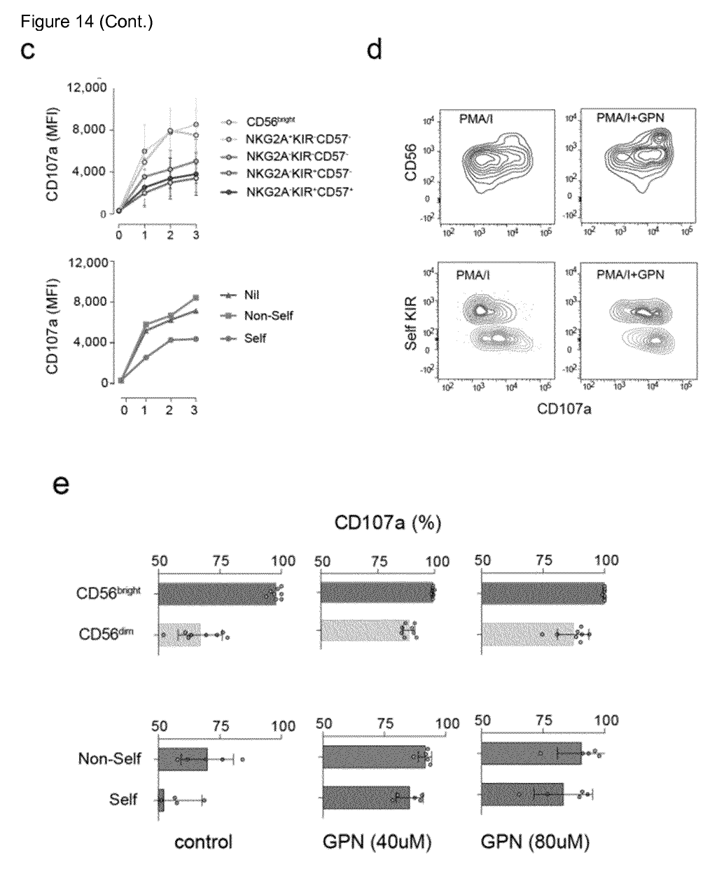

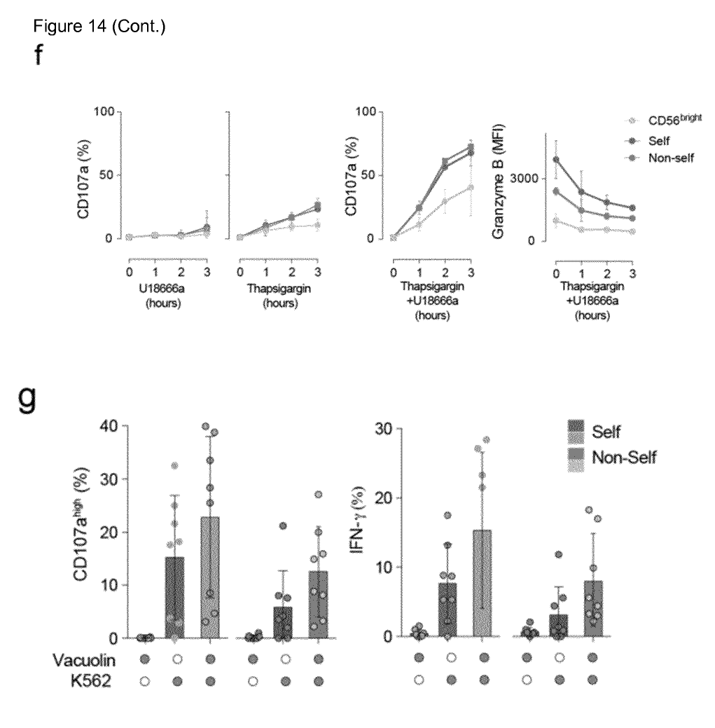

FIG. 14 shows that the secretory lysosome functions as a signalling hub in NK cells. In A a representative example of granzyme B and CD107a expression following stimulation of NK cells with K562 cells in the presence or absence of GPN is shown. B shows the frequency of formation of CD107^{High+} (top) and IFN-γ⁺ (bottom) from Self and Non-Self KIR⁺ NK cells following stimulation with K562 cells in the presence or absence of 50 uM GPN (left), 10 uM mefloquine (middle) or 10 uM siramesine (right). In C a representative example is shown of CD107a up-regulation in response to PMA/Ionomycin exposure over time versus differentiation (top) and education (bottom). D shows representative plots and E a summary of CD107a responses in CD56^{bright} and CD56^{dim} NK cell subsets (top) and Self and Non-Self KIR⁺ NK cells (bottom) following stimulation with K562 cells in the presence of the indicated doses of GPN. F shows mobilization of CD107a (left three panels) and loss of granzyme B (far right panel) over time in the indicated subset following stimulation with U18666 or thapsigargin alone (left panels) or in combination (right panels). G shows the frequency of formation of CD107a^{High+} (left) and IFN-γ⁺ NK cells (right) after stimulation with K562 cells in the presence of 10 μM vacuolin-1. H shows the relative phosphorylation of the indicated signalling molecules following stimulation with biotinylated anti-CD16 (10 μg/mL) cross-linked with avidin in the presence of 50 μM GPN or 10 μM vacuolin-1.

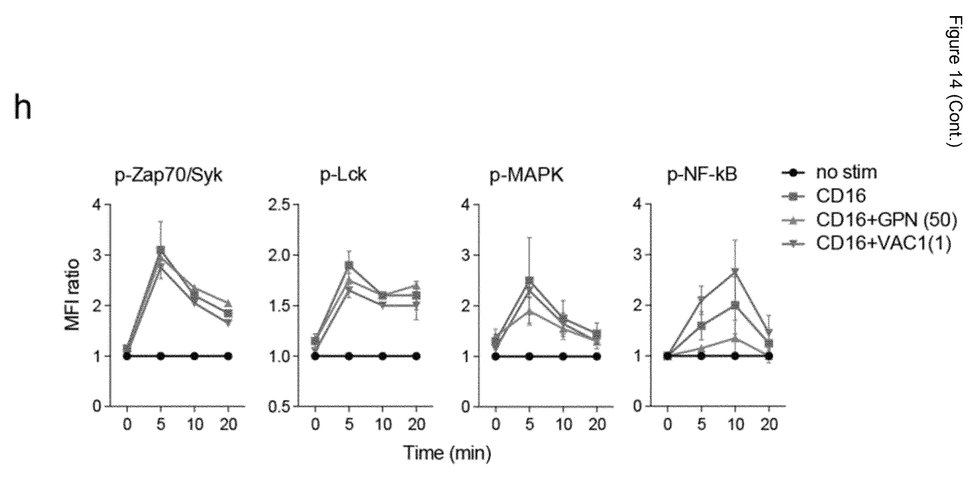

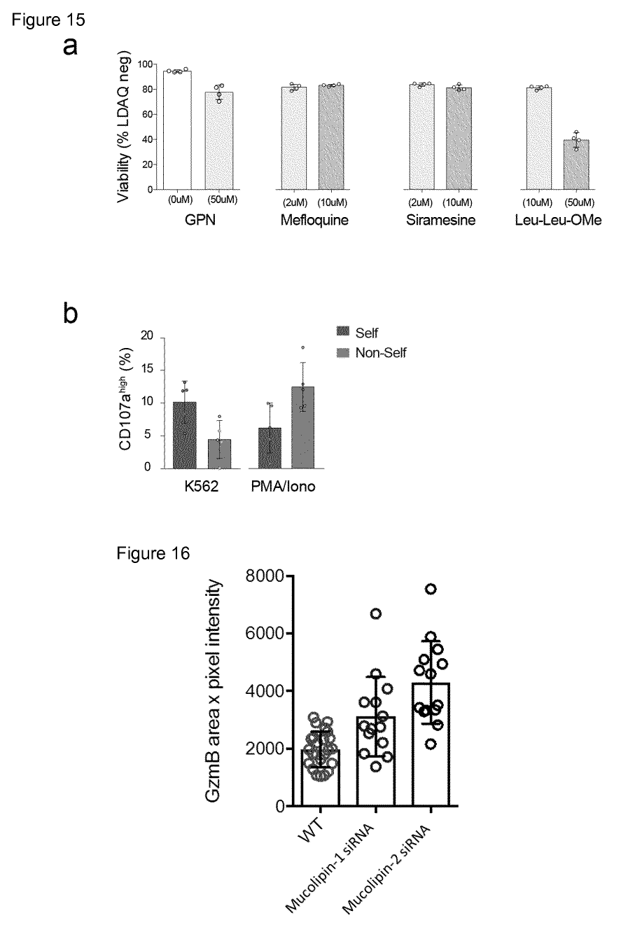

FIG. 15 shows that the secretory lysosome functions as a signalling hub in NK cells. A shows the viability of NK cells exposed to the indicated concentrations of lysosomotropic compounds. B shows the frequency of formation of CD107^{high+} NK cell subsets in response to either K562 target cells or phorbol 12-myristate 13-acetate (PMA)/lonomycin in self- and non-self-specific NK cells (n=5).

FIG. 16 shows that siRNA silencing of TRPML1 and TRPML2 in primary NK cells leads to increased accumulation of GzmB.

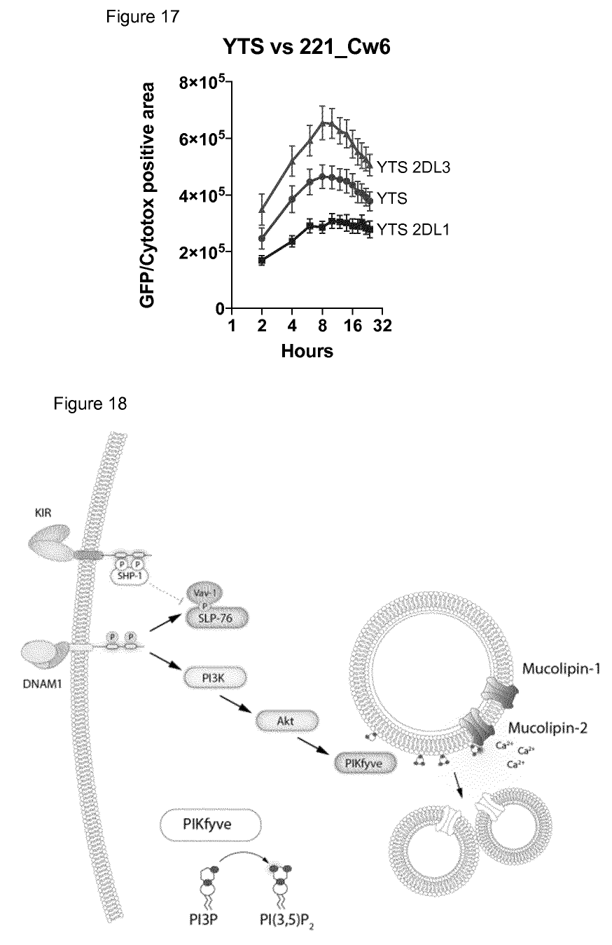

FIG. 17 shows the cytolytic activity of YTS NK cells against 221 cells expressing the HLA-C2 ligand HLA-Cw6. Introduction of a self-KIR receptor (2DL3) into the YTS NK cell line leads to increased killing of 221-Cw6 cells. Notably, YTS 2DL1 cells are turned off by the cognate ligand HLA-Cw6.

FIG. 18 is a schematic diagram demonstrating a newly-elucidated signalling pathway whereby, in the absence of signalling from inhibitor receptors, weak agonistic input through NK cell activating receptors initiates PI3K-dependent phosphorylation of Akt. Akt in turn phosphorylates and activates PIKfyve. PIKfyve, when activated, phosphorylates phosphatidylinositol-3-phosphate to phosphatidylinositol-3,5-bisphosphate, leading to activation of TRPML1 and TRPML2. TPML1 and TRPML2 drive lysosomal fission, leading to loss of NK cell functional potential.

### EXAMPLES

### Example 1—Activation Thresholds in Natural Killer Cells Determined by Receptor-Regulated Granular Potential

### Methods

### Cells

Buffy coats from random healthy blood donors were obtained from the Karolinska University Hospital and Oslo University Hospital blood banks with informed consent. Peripheral blood mononuclear cells were separated from buffy coats by density gravity centrifugation (Lymphoprep; Axis-Shield) using fretted spin tubes (Sepmate; Stemcell Technologies). Genomic DNA was isolated from 200 μl of whole blood using DNeasy Blood and Tissue Kit (Qiagen). KIR ligands were determined using the KIR HLA ligand kit (Olerup SSP) for detection of the HLA-Bw4, HLA-C1, and HLA-C2 motifs. NK cells were purified using negative selection (Miltenyi) with an AutoMACS Pro Seperator. K562 cells were maintained in RPMI+10% FCS.

### Antibodies, Flow Cytometry and FACS Sorting

Isolated PBMC were stained for flow cytometric analysis using an appropriate combination of the following antibodies, followed by the name of the clone in brackets: CD14-V500 (M5E2), CD19-V500 (HIB19), CD3-V500 (UCHT1), CD56 ECD (N901) CD57-PB (HCD57), CD57 purified (TB01), anti-mouse-IgM-EF650 (II/41), NKG2A-PE, APC or APC.AF750 (Z199), CD16-BV785 (3G8), KIR2DL3-FITC, KIR2DL1-APC or APC-Vio770 (REA284), KIR3DL1-AF700 or BV421 (D×9), KIR2DS4-QD585 (1847), KIR3DL2-biotin (D×31), KIR2DL2/L3/S2-PE.Cy5.5 (GL183), KIR2DL1/S1-PE.Cy7 (EB6) or PE-Vio770 (11 PB6). Dead cells were labelled using live/dead aqua (Life technologies). Biotin-conjugated antibodies were visualized using streptavidin-Qdot 585 or 605 (Life technologies). After surface staining, cells were fixed and permeabilized using a fixation/permeabilization kit (BD Bioscience Cytofix/Cytoperm) prior to intracellular staining with anti-granzyme B-A700 (GB11). Samples were acquired using an LSRII flow cytometer (Becton Dickinson) and data was analysed using FlowJo V10.0.8 (TreeStar).

Purified NK cells were stained for sorting using the following combination; CD56-ECD, CD57-FITC, NKG2A-PE, KIR2DL1-APC-Vio770, KIR2DL1/S1-PE-Vio770, KIR3DL1/S1-APC (Z27.7.3), KIR2DL2/L3/S2-PE.Cy5.5 (GL183). Cells were sorted using a FacsARIA at 4° C. (BD).

NK cells of the cell line YTS were modified to express the self-KIR receptor 2DL3, which recognises HLA-C alleles including HLA-C1, which is expressed by YTS NK cells. As a control, YTS NK cells modified to express the non-self-KIR receptor 2DL1 (which recognises HLA-C4) were used. The modified YTS NK cells were stained, fixed and permeabilised as above. Intracellular staining was then performed using anti-granzyme B-A700 (GB11), anti-granzyme A or anti-perforin antibodies. Samples were then analysed as above.

### Stochastic Neighbour Embedding (SNE) Analysis

FCS files from all donors were imported into FlowJo version 10.0.8 (TreeStar) and NK cells were identified based on gating for CD3(−) and CD56(+) expression. These events were exported as FCS for further processing using R version 3.1.0. 5000 events were randomly sampled from each file and the individual donors were then pooled for analysis. Two-dimensional Barnes-Hut t-distributed SNE was performed with the Rtsne R package (http://cran.r-project.org/package=Rtsne). The SNE calculation was based on the markers KIR2DL1, KIR2DL3, KIR3DL1, KIR3DL2 and NKG2A and plots were generated using the ggplot2 R package (http://ggplot2.org). Red borders indicating the population were added using Photoshop CS6 (Adobe).

RNAseq and qPCR

RNASeq was performed using single-cell tagged reverse transcription (STRT), a highly multiplexed method for single-cell RNA-seq. Real time quantitative PCR was used to study the difference in the expression of 20 genes of interest in sorted differentiation and education subsets of NK cells. RNA was isolated using RNeasy mini kit (Qiagen). Following RNA isolation, cDNA was synthesized using First strand synthesis kit (Qiagen) according to the manufacturer's protocol. Customized RT² Profiler PCR array (Qiagen) was ordered with specific primers for the 20 genes of interest, as well as 2 housekeeping genes, a reverse transcriptase control, genomic DNA control, and a positive PCR control. Real time quantitative PCR was performed on cDNA from differentiation and education subsets and the data obtained was normalized using 18S rRNA and B2M as housekeeping genes. All target genes were run as triplicates and analysis of qPCR data was done using qBase+ (Biogazelle).

### Confocal Fluorescence Microscopy and Image Analysis

Sorted NK cells were prepared for confocal microscopy using fixation/permeabilization (BD Bioscience Cytofix/Cytoperm) prior to intracellular staining with mouse anti-human granzyme B-A647 (GB11), rabbit anti-human pericentrin (Ab4448) followed by Donkey anti-Rabbit IgG Alexa555 and donkey anti-mouse-A568. After staining, the fixed cells were adhered to glass cover slips using Cell Tak (Corning) and mounted using Pro-long Gold Antifade with DAPI. The cells were examined with a Zeiss LSM 710 confocal microscope (Carl Zeiss Microlmaging GmbH) equipped with an Ar-Laser Multiline (458/488/514 nm), a DPSS-561 10 (561 nm), a Laser diode 405-30 CW (405 nm), and a HeNe-laser (633 nm). The objective used was a Zeiss plan-Apochromat 63×NA/1.4 oil DICII. Image processing and analysis were performed with basic software ZEN 2011 (Carl Zeiss Microlmaging GmbH) and Imaris 7.7.2 (Bitplane AG). Confocal z-stacks were deconvolved using Huygens Essential 14.06 (Scientific Volume Imaging b.v.), and visualized with Imaris.

For the PIKfyve inhibition experiments, fresh NK cells were left either untreated or cultured in RPMI/10% FCS, supplemented with 1 μM Vacuolin-1, YM201636 or Apilimod at 37° C. in 5% CO₂ overnight. Cells were surface-stained with anti-human HLA-ABC antibody (W6/32, BioLegend) prior to settlement on CelITak-coated slides for 20 minutes, followed by fixation with 4% paraformaldehyde. Intracellular staining was performed by incubation with anti-human LAMP-1 (Ab24170, Abcam) for 4h, followed by donkey anti-rabbit-A488 antibody (abcam) and donkey anti-mouse-A568 antibody (Abcam) and Hoechst33342.

Where indicated, cells were stained with mouse anti-human granzyme B-A647 (GB11) instead of anti-HLA-ABC, followed by donkey anti-mouse-A568.

### Electron Microscopy

Sorted NK cells for immuno-EM were fixed in a mixture of 4% formaldehyde and 0.1% glutaraldehyde in 0.1 M PHEM buffer (60 mM PIPES, 25 mM HEPES, 10 mM EGTA and 2 mM MgCl₂ at pH 6.9), followed by embedding in 10% gelatine, infiltration with 2.3 M sucrose and frozen in Liquid N₂. Ultra-thin sections (70-90 nm) were cut on a Leica Ultracut (equipped with UFC cryochamber) at −110° C., picked up with a 50:50 mixture of 2.3 M sucrose and 2% methyl cellulose. Sections were then labelled with antibodies against granzyme B (496B, eBioscience) or Chondroitin Sulphate 4 (2B6, AMSBIO), followed by a bridging rabbit-anti-mouse antibody (DAKO, Denmark) and protein A gold (University Medical Center, Utrecht, Netherlands). Microscopy was done at 80 kV in a JEOL_JEM1230 and images acquired with a Morada camera. Further image processing was done in Adobe Photoshop. Quantification was done according to established stereological procedures.

### Functional Assays

Functional assays were performed at 37° C. in complete medium (RPMI+10% FCS) for the times indicated. Purified NK cells were incubated with K562 target cells for 5 hours at a ratio of 1:1 in the presence of anti-CD107a-FITC (biolegend) for degranulation assays, or with the addition of Brefeldin A (GolgiPlug BD) for degranulation plus intracellular cytokine assays. Treatment with lysosomotropic reagents were performed for the duration of the assay using the following final concentrations; glycyl-L-phenylalanine-β-naphthylamide (GPN, 50 μM), mefloquine (10 μM), siramesine (10 μM), vacuolin-1 (10 μM). Stimulation with PMA (1 μg/mL) and ionomycin (0.5 μM), or with thapsigargin (1 μM) and/or U18666a (2 μg/mL) were performed for the times indicated, and the cells were stained for CD107a surface expression post-stimulation.

### Phospho-Flow Cytometry

Functional assays for phospho-flow cytometry were performed at 37° C. in complete medium in NK cell suspensions between 5-10 M/mL for 20 min. Cells were pre-treated for 1 h using GPN (50 μM) or vacuolin-1 (10 μM), after which biotinylated CD16 (Biolegend, clone 3G8) was added to final concentrations of 5 μg/mL each. After 1 min, the aliquot for the 0 min (unstimulated) sample was taken out and mixed with Fix Buffer I (BD Biosciences). After one additional minute, the stimulation was started by crosslinking the biotinylated antibodies via 50 μg/mL avidin (Thermo Fischer Scientific) and the aliquots for the 5 min, 10 min and 20 min samples were transferred into Fix Buffer I (BD bioscience) at the corresponding time points. Cells were fixed at 37° C. for 10 min, washed and re-suspended in PBS. To allow combination of the differently stimulated samples into one, two dimensional fluorescent cell barcoding (FCB) was utilized. Samples were stained in distinct concentrations of amine-reactive pacific blue succinimidyl ester (Thermo Fisher Scientific) for the time points (0 min-0.69 ng/mL, 5 min-6.25 ng/mL, 10 min-25 ng/mL and 20 min-100 ng/mL) in combination with amine-reactive pacific orange succinimidyl ester (Thermo Fisher Scientific) for the different stimulations (control—10 ng/mL, GPN—100 ng/mL and vacuolin-1-500 ng/mL). After 20 min at RT, samples were washed twice in wash solution (PBS supplemented with 1% FCS and 0.09% sodium azide), combined, permeabilized (Perm Buffer III, BD Biosciences) and stored at −80° C. For thawing, samples were incubated 20 min on ice. Then, they were washed in wash solution and stained with Alexa Fluor 647-conjugated phospho epitope-specific antibodies against ZAP70/syk (pY319/pY352), Lck (pY505), Erk1/2 (pT202/pY204) (BD Bioscience), NF-κB p65 (pS536) (Cell Signaling Technologies) or isotype control IgG1K (BD Biosciences) for 30 min at RT. After washing data was acquired on an LSR Fortessa (BD Biosciences) and analysed in with FlowJo v10.0.8 (TreeStar).

### si RNA Knockdown

PBMC were purified from buffy coats using ficoll gradient. NK cells were isolated from purified PBMC using Miltenyi NK cell Isolation kit. Isolated NK cells were stimulated in the presence of 10 ng/mL IL15 for 3 days, harvested and transfected with 300 pM siRNA (Dharmacon siRNA ONTARGET SMARTPOOL of target gene or control) using an Amaxa Nucleofector and Lonza Macrophage transfection kit. The cells were rested in OptiMEM for 2 hours post-transfection and then transferred to culture medium+1 ng/mL IL15 fora further 48-72 hours. siRNA knockdown was confirmed using real time quantitative PCR analysis. Granzyme B phenotypes were analysed by flow cytometry as described above.

### Statistical Analysis

Comparisons of matched groups were made using paired Students T test. Single comparison of groups or populations of cells between donors was performed using Students t test or Mann-Whitney test for statistical significance. n.s. indicates not significant; ***p<0.001; **p<0.01; and *p<0.05. Analyses were performed using GraphPad Prism software.

### Results

Self-Recognition is Associated with Increasing Granular Load in Primary Resting NK Cells.

In order to address the mechanisms involved in calibration of effector potential in NK cells, the expression of cytotoxic effector molecules was monitored in discrete subsets of resting NK cells by flow cytometry. The expression of granzyme B and perforin increased gradually whereas granulysin decreased with NK cell differentiation, from CD56^{bright} NK cells through discrete stages defined by the expression of NKG2A, KIR and CD57 (FIG. 1A-C and FIG. 2A). Next, granzyme B content in mature NK cells was stratified based on the expression of self-versus non-self-specific KIR. Clustering of NK cell phenotypes using t-distributed stochastic neighbour embedding (tSNE) revealed high expression of granzyme B in NK cell subsets expressing self-specific KIR (FIG. 1D). Extended analysis in 64 healthy donors showed significantly higher expression of granzyme B in NK cells expressing one or more self-specific KIR a in donors homozygous for HLA-C1/C1 or HLA-C2/C2, the respective ligands for KIR2DL3 (2DL3) and 2DL1 (FIGS. 1E & F). Similar results were observed for perforin and granulysin that were both expressed at higher levels in self-specific NK cells (FIG. 2B). Corroborating the link between inhibitory input through self-KIR and granzyme B expression, donors that were heterozygous for HLA-C1/C2 had similar and high levels of granzyme B in 2DL1 and 2DL3 single-positive NK cells (FIG. 1F). Granzyme B expression was also high in 3DL1⁺ NK cells from donors positive for its cognate ligand HLA-Bw4 (FIG. 1G) and varied with respect to the expression level of 3DL1 (FIG. 1H). NK cells with higher levels of 3DL1 surface expression, also known to have a higher functional capacity, exhibited greater expression of granzyme B than those with low expression. Notably, this phenotype was only observed in donors harbouring the cognate HLA-Bw4 ligand (FIG. 1I).

Similarly, NK cells of the cell line YTS, modified to express the self-KIR receptor 2DL3 showed significant increases in granzyme B and perforin content, and a slight increase in Granzyme A content, relative to YTS NK cells modified to express the non-self-KIR receptor 2DL1 (FIG. 3).

These data show that inhibitory self-KIR interactions are tightly connected to the granular load (specifically that inhibitory self-KIR interactions drive an increase in granular load), establishing an important link between inhibitory input and the core cytolytic machinery of NK cells.

### Inhibitory Input Influences the Level of Granular Content Independently of Transcriptional Cell Differentiation Programs.

To address whether the increased levels of granzyme B in educated NK cells was due to gene expression, we studied the transcriptional regulation of effector programs in the context of NK cell differentiation. Transcriptome analysis was performed by using single-cell tagged reverse transcription (STRT), a highly multiplexed method for single-cell RNA sequencing (RNA-Seq), on naïve CD56^{bright} NK cells and five CD56^{dim} NK cell subsets, sorted on the expression of NKG2A and KIR. Since the exact order of transitions between intermediate stages of CD56^{dim} NK cell differentiation are not known, we focused our analysis on transcription factors linked to the regulation of GzmB in three of these five discrete subsets representing previously defined stages of NK cell differentiation: CD56^{bright}, CD56^{dim} NKG2A⁻KIR⁻ and CD56^{dim} NKG2A⁻KIR⁺ NK cells. CD56^{bright} NK cells predominantly expressed higher levels of transcription factors associated with induced responsiveness to acute stimulation such as API, STAT-1, STAT-5 and NF-κB. The more mature CD56^{dim} NK cell subsets, on the other hand, expressed higher levels of transcription factors associated with cellular differentiation, including TBX21, EOMES, and those more recently found to be associated with maintenance of effector expression, such as BATF, IRF4, NFATc1 and PRDM1 (FIG. 4A). Although there is limited data on their role in human NK cell differentiation, TBX21, PRDM1 and IRF4 have been previously associated with transcription of GzmB, perforin and IFN-γ. The expression of IRF4 was observed to increase, at both the transcriptional and protein level, with differentiation and acquisition of KIR (FIGS. 4A & B). Increasing expression of IRF4 correlated with the level of granzyme B protein expression in the same subsets of human NK cells (FIG. 4C).

We next addressed whether transcriptional changes in effector loci associated with differentiation and KIR acquisition accounted for the observed differences in granzyme B content between self- and non-self-specific NK cell subsets. NKG2A⁻CD57⁻ NK cells were sorted by FACS into single 2DL3 or 2DL1 positive populations from C1/C1 and C2/C2 donors and tested against a panel of 20 selected qPCR targets comprising transcription factors and canonical cell surface markers linked to NK cell differentiation, GzmB regulation and granule biogenesis (FIG. 4D and FIG. 5). For the qPCR targets that displayed clear and predictable trends between the CD56^{bright} and CD56^{dim} NK cell subsets based on differentiation, we found no corresponding difference in granzyme B mRNA expression between the sorted self- and non-self-specific NK cell subsets. These data demonstrated that the increased granular loading in educated NK cells was independent of gene expression.

In mouse NK cells, expression of granzyme B is further regulated by cytokine-induced translation from a pre-existing pool of mRNA transcript. Therefore, we explored the possibility that self- and non-self-specific NK cells respond differentially to cytokine priming in vivo, resulting in divergent steady-state levels of expressed granzyme B. To address this possibility, NK cells exposed to IL-15 or IL-21 for various lengths of time were monitored for granzyme B content using flow cytometry (FIG. 4E). CD56^{bright} NK cells responded to priming by IL-15, but not to IL-21, in agreement with the dominant expression of STAT-5A in this subset (FIGS. 4A & E). In contrast, CD56^{dim} NK cells, expressing both STAT3 and STAT-5B, displayed corresponding increases in the level of granzyme B in response to both IL-15 and IL-21 stimulation. Notably, the relative difference in granzyme B between self- and non-self-specific NKG2A⁻CD57⁻ NK cells was similar after stimulation with IL-15 or IL-21 (FIG. 4F). Furthermore, blockade of STAT-5 and mTOR signalling using Pimozide and Torin-1, respectively, reversed the cytokine-induced increase in granzyme B in both self- and non-self-specific NK cells (FIG. 4G). Importantly, the same treatment with Pimozide and Torin-1 did not further reduce the pre-existing constitutive levels of granzyme B that preceded cytokine stimulation. Similar effects were noted with the Janus kinase inhibitor Ruxolitinib and the STAT3 inhibitor S3I-201 (FIG. 6). This suggests that the observed differences in granzyme B levels at rest are stable and refractory to interference of either STAT or mTOR signalling.

### Educated NK Cells Retain Primed Granzyme B in Large Granular Structures Near the Centrosome.

Granzyme B is sequestered into granular structures within the cell. To determine whether the increased levels of granzyme B was a result of higher density, number, or size of cytolytic granules in self-specific NK cells, or a combination thereof, NKG2A⁻CD57⁻ NK cell subsets were sorted ex vivo into self- or non-self-specific NK cell subsets and imaged by confocal microscopy. Corroborating the difference in granzyme B expression observed using flow cytometry, educated NK cells had higher overall intensity of granzyme B staining, localized within granular structures (FIG. 7A and FIG. 8). The average number of granzyme B⁺ granules, as defined by discrete points of localized staining intensity, was similar between self- and non-self-specific NK cells. However, educated NK cells displayed an increasing level of fluorescence intensity for granzyme B in the granular areas, which in turn correlated with proximity to the centrosome (FIG. 7B).

Optical resolution limits of confocal microscopy prevented accurate resolution of granule size and determination of areas with high granzyme B intensity. To address more precisely the size and density of individual granules, sorted self-KIR⁺ and non-self KIR⁺ NK cells were sectioned and stained for immuno-electron microscopy (Immuno-EM) using anti-granzyme B monoclonal antibody and gold-particle labelled Protein A (FIG. 7C). Quantification of gold particles per cellular section revealed overall greater granzyme B staining in educated NK cells, consistent with both the flow cytometry and confocal data (FIG. 7D). Notably, the average size of granules was larger in educated NK cells resulting in larger total granular area (FIGS. 7E & F), without a general increase in relative cell size (FIG. 9A). Next, we plotted the distribution of cells as a function of their granular areas. While both self- and non-self KIR⁺ NK cell subsets contained cells covering the entire spectrum of net granular area, self-KIR′ NK cells had a higher fraction of cells containing individual granules with areas above approximately 0.4 μm² (FIG. 7G). Notably, it was only among cells with larger individual granular areas that we found granzyme B-dense granules (FIG. 7H). To further examine the granular composition, we stained sections of self-KIR′ and non-self KIR⁺ NK cells with Chondroitin Sulphate 4 (CS4), a predominant glycosaminoglycan side-chain associated with serglycin in cytotoxic lymphocytes. Serglycin is critical for retention of granzyme B in cytotoxic granules, resulting in NK cells with hypofunctional responses in serglycin^{-/-} mice. Similar to granzyme B, we found a higher intensity of CS4 in self-KIR′ NK cells (FIG. 71 and FIG. 9B-E). Thus, the data provide a link between expression of self-specific inhibitory KIR and retention of large granzyme B-dense cytotoxic granules.

Treatment of primary NK cells with the PIKfyve inhibitors YM201636, apilimod or vacuolin-1 was found to drive an increase in secretory lysosome size (FIG. 10), and vacuolin-1 treatment was also demonstrated to cause granzyme B to cluster within the large secretory lysosomes formed (FIG. 11).

Self-KIR⁺ NK Cells Display High Intensity of CD107a Following Target Stimulation and is Associated with Release of Large Granules and Loss of Granzyme B.

For all imaging approaches used to characterize educated NK cells at the morphological level, the proportion of cells containing large, granzyme B-dense structures was variable. This raised the question as to whether the morphological phenotype correlates with intrinsic functional potential. The state of education for a given NK cell subset has typically been determined experimentally by its overall responsiveness to stimulation. However, even within well-defined single-KIR′ NK cell subsets the responses are highly variable, with only a fraction of cells responding at any given time-point. Here, close examination of granule mobilization in self-versus non-self-specific KIR⁺ NK cells following stimulation with K562 cells revealed that CD107a responses were dominantly observed in granzyme B^{high} educated NK cells (FIGS. 12A & B). Furthermore, the loss of granzyme B following degranulation was associated with intense surface staining of CD107a (FIG. 12C). Hence, the low levels of CD107a expression observed in non-self KIR⁺ NK cells was not associated with a loss in granzyme B, suggesting that surfacing of CD107a was not linked to granular release in this subset. Supporting this notion, immuno-EM of sorted CD107a^{high} NK cells revealed a reduction of large and granzyme B-rich granules (FIGS. 12D & E).

This was confirmed by the modification of the YTS NK cell line with either the self-KIR 2DL3 or the non-self-KIR 2DL1. YTS NK cells modified with the self-KIR 2DL3 demonstrated much greater CD107a responses (indicating increased functionality) against target 221 cells than did unmodified YTS NK cells or YTS NK cells modified with the non-self-KIR 2DL1 (FIG. 13).

Together these data show that the expression of self-KIR is associated with the accumulation of large and granzyme B-dense granules and that this unique morphological feature is linked to quantitatively greater release of granzyme B following target cell conjugation.

The Lysosome as a Programmable Signalling Hub that Tunes NK Cell Function.

Intracellular communication between organelles has recently emerged as a critical component in the mobilization and propagation of intracellular Ca²⁺ signals and endolysosomal traffic. The relative capacity of the acidic compartment, which includes the granular compartment, in terms of uptake and release of intracellular Ca²⁺ bears important implications for NK cell functions including exocytosis and cytokine production. Given the difference in granularity between self- and non-self-specific NK cells, we sought to establish whether the capacity of the granular compartment is involved in modulating NK cell function through its effect on intracellular Ca²⁺ signalling.

First, functional responses were determined from primary NK cells in the presence and absence of glycyl-L-phenylalanine-beta-naphthylamide (GPN), a lysosomotropic dipeptide substrate of cathepsin C. Treatment with GPN causes osmotic permeabilization of lysosomal membranes, resulting in equilibration of small solutes, including Ca²⁺, between the acidic compartment and the cytosol (e.g. loss of Donnan potential). Not only did treatment with GPN abrogate specific degranulation in self KIR⁺ NK cells in response to K562 cells, it also reduced corresponding IFN-γ expression (FIGS. 14A & B). Similar results were obtained using two alternative lysosomotropic agents, Mefloquine and Siramesine (FIG. 14B), revealing that disruption of the granular compartment affects both the mobilization of cytolytic granules and the production of cytokine, responses that are both Ca²⁺ dependent. Importantly, none of these compounds showed any general cellular toxicity at the doses tested compared to the positive control L-Leucyl-L-leucine methyl ester (LeuLeuOMe) that is a lysosomotropic agent known to induce apoptosis in immune cells (FIG. 15A).

To further explore the interplay between the granular compartment and Ca²⁺ signalling, NK cells were treated with PMA and lonomycin (PMA/I). Together, PMA and lonomycin bypass receptor tyrosine kinase mediated phosphorylation of PLC-γ, resulting in direct activation of Protein Kinase C (PKC)⁴⁶ and elevation of cytosolic Ca²⁺, leading to stimulation of downstream effector responses. In contrast to stimulation with K562 target cells, PMA/I induced mobilization of CD107a with reversed kinetics, being faster and more pronounced in less granular cells such as the CD56^{bright} NK cells (FIG. 14C). This delay and decrease in magnitude of granule mobilization was also observed for self KIR⁺ in comparison to non-self KIR⁺ NK cell subsets (FIG. 14C and FIG. 15B).

Interference with Ca²⁺ uptake by the acidic compartment has previously been demonstrated to enhance Ca²⁺ signalling in HEK cells, suggesting that this compartment is able to sequester cytosolic Ca²⁺ and thereby, together with the ER, buffer cytosolic Ca²⁺ levels and increase the threshold for activation. Indeed, disruption with GPN reversed the higher threshold for PMA/I-induced activation in self-specific NK cells (FIGS. 14D & E). Selective blockade of Ca²⁺ uptake by the ER using SERCA inhibitor thapsigargin, or by the acidic compartment using the cationic amphiphile U18666a, was used to examine the relative contribution of these compartments in the buffering of cytosolic Ca²⁺ (FIG. 14F). The combination of these two compounds was enough to trigger strong spontaneous mobilization of the granular compartment in otherwise unstimulated cells, suggesting that a rise in cytosolic Ca²⁺ through inhibition of intracellular Ca²⁺ uptake was sufficient to provoke granule mobilization. The rate of mobilization under these conditions was constant with respect to self-versus non-self-specific NK cells, suggesting the capacity for mobilization is equivalent in each of the different NK cell populations when the effect of the granular compartment on uptake of Ca²⁺ is subverted. Again, this suggests the mobilization of effector functions is affected by the differential uptake of calcium by the granular compartment in self-specific NK cells. These data collectively corroborate the notion that the large cytotoxic granules in self KIR⁺ NK cells may serve as a signalling hub that can raise the threshold for response to Ca²⁺ flux initiated outside of the acidic compartment, whilst also potentiating responses that directly stimulate the granular compartment to release Ca²⁺.

Next, we set out to examine whether the acidic compartment could serve as a programmable organelle to induce gain of function in NK cells. The small chemical vacuolin-1 is used to recapitulate Chediak-Higashi Syndrome (CHS) in vitro through homotypic fusion of lysosomes. In CHS, mutation of the CHS1 gene leads to the formation of giant secretory lysosomal structures. Vacuolin-1 was recently shown to inhibit the PI3P 5-kinase PIKfyve, which produces PtdIns (3,5) P2. This lipid product is known to control endosome fusion in part by activating a lysosomal Ca²⁺ channel, TRPML1. Incubation of resting NK cells with vacuolin-1 alone had no effect on NK cell function. However, treatment with vacuolin-1 followed by stimulation with K562 target cells enhanced the degranulation and IFN-γ response in both self and non-self NK cell subsets (FIG. 14G). This lowered threshold for activation suggests that homotypic fusion within the acidic compartment may play a role in promoting effector functions in the short term.

In order to identify the point at which the granular compartment intersects with intracellular signalling pathways, phospho-flow cytometry was performed in combination with chemical modulation (both positive and negative) of the acidic compartment in primary NK cells. This allowed us to probe signalling both proximal and distal of the plasma membrane. GPN and vacuolin-1 had a minimal effect on upstream signalling including ZAP70 and Lck following ligation of CD16 (FIG. 14H). However, the propagation of down-stream signals, in particular through NF-κB, were decreased by treatment with GPN and increased by treatment with vacuolin-1. These data demonstrate a role for the cytolytic granules in propagating downstream activation signals in NK cells, having important consequences for functionality, including degranulation and cytokine production.

### TRPML1/2 Knockdown Enhances NK Cell Functionality

TRPML1 and TRPML2 are cation channels located in the lysosomal membrane. Their activity is indirectly up-regulated by PIKfyve. Knockdown of TRPML1 and TRPML2 expression in primary NK cells using siRNA was found to cause the GzmB content of the cells to increase, enhancing their functionality (FIG. 16).

### NK Cell Activity is Altered by Inhibitory Receptor Expression

YTS NK cells were modified to express either the self-KIR 2DL3 or the non-self-KIR 2DL1. YTS-2DL3, YTS-2DL1 and unmodified TS NK cells were incubated with target 221 cells, which natively express HLA-Cw6, which is the cognate ligand of the 2DL1 KIR receptor. As shown in FIG. 17, NK cells expressing the self-KIR 2DL3 were found to display enhanced activity against the 221 cells, relative to the unmodified YTS cells. Cells expressing the 2DL1 KIR receptor were found to display reduced activity against the 221 cells relative to the unmodified YTS NK cells, due to HLA-Cw6-mediated signalling through the 2DL1 receptor.

### Discussion

The cellular and molecular mechanisms that connect inhibitory signalling to global gain of function in NK cells are incompletely defined. We explored the effects of interactions between inhibitory receptors and self-MHC class I molecules on NK cell morphology and discovered that the gain of function in self-specific NK cells was associated with accumulation of large, granzyme B-dense granules. This difference in granularity between self- and non-self-specific NK cells can be observed readily ex vivo and appears to be independent of steady state transcriptional programmes that drive lysosomal biogenesis, cellular metabolism and the expression of effector loci. Lysosomotropic agents that disrupt lysosomes were found to down-modulate NK cell responses whereas homotypic fusion of lysosomes by small chemicals led to gain of function. A major implication of these findings is that the granular compartment itself behaves as a signalling hub that regulates the threshold for NK cell activation. A close connection between granular loading and expression of self-specific inhibitory receptors would allow effector cells to circulate in a stabilized, highly functional pre-primed state in a manner that is temporally distinct from priming and eliminates the need for continuous stimulation to maintain the effector phenotype.

A variety of models and a vast nomenclature have been used to describe the process of NK cell education. However, regardless of whether the functional phenotype is caused by gain of function (arming) in educated NK cells or loss of function (disarming) in hypo-responsive NK cells, the net outcome is generally interpreted in terms of differential thresholds for NK cell activation. A structural basis for the difference in activation threshold was recently proposed. Guia et al. Sci Signal 4, ra21 (2011), noted that educated NK cells display a unique compartmentalization of activatory and inhibitory receptors at the nano-scale level at the plasma membrane. Notably, these ultra-structural differences correlated with the magnitude of the Ca²⁺ flux following ligation of the activating receptors, suggesting that the spatial organization of the receptors into areas which are differently permissive to signalling may contribute to setting the threshold for activation in NK cells. Extending these findings, we show that the low threshold for activation in self-KIR′ NK cells is also tightly linked to the presence of large, granzyme B-dense granular structures, which are not found in corresponding non-self KIR⁺ NK cells. This led us to speculate that the granules themselves may accumulate functional potential, and that the retention of these structures is under the influence of inhibitory interactions with self HLA class I.

In T-cells, cytolytic granules serve as acidic Ca²⁺ stores that are mobilized by nicotinic acid adenine dinucleotide phosphate (NAADP) acting on two-pore channels (TPCs) on the membrane of the granules themselves. Qualitative differences between signals that are channelled through the acidic compartment versus those that directly activate depletion-dependent Ca²⁺ induced calcium release (CICR) by the ER suggested a critical role for local Ca²⁺ microdomains involving TPCs that stimulate granule exocytosis. These data support the previously described “trigger hypothesis”, suggesting that second messengers that stimulate release from the acidic compartment, including NAADP and cyclic adenosine diphosphate ribose (cADPR), trigger an initial burst of Ca²⁺ that is subsequently amplified by Ca²⁺ release from the ER. Our data reveal the possibility of a quantitative relationship between accumulation of large granular structures and intrinsic functional potential connected to the differential capacity to uptake and release Ca²⁺ in educated NK cells. We propose that similar fine-tuning and retention of T-cell functional potential may also be achieved through modulation of granular morphology in subsets of T-cells, possibly through the action of inhibitory receptors such as KIR, CTLA-4 and PD-1.

NK cell education is reflected in a spectrum of functional outcomes including degranulation and cytokine production. Release of cytolytic granules and the release of cytokine each represent distinct secretory pathways, yet both are reflected by NK cell education. An increasing body of evidence supports the role of the acidic compartment not only in the triggering and elaboration of calcium signalling, but also in the spatiotemporal coordination of signalling cascades. In both T-cells and NK cells, lysosomal calcium release plays an important role in degranulation. This is illustrated in patients with Niemann-Pick disease type C, a lysosomal storage disorder based on a mutation in the NPC1 gene, in which defects in the signalling from the acidic compartment affect downstream effector functions. NPC1 protein is involved in the efflux of sphingosine from the lysosome. When disrupted, the Ca²⁺ stores fail to refill leading to depletion of Ca²⁺ from the lysosome and loss of signalling in NK cells. The role of the acidic compartment in promoting the translocation of transcription factors has more recently come to light with the demonstration that nuclear translocation of the transcription factor TFEB, an important driver of lysosomal biogenesis and autophagy, was dependent on lysosomal Ca²⁺ release in a calcineurin-dependent manner. Given the pericentrosomal clustering that occurs during granule polarization, it is possible that a similar relationship may exist between acidic release of Ca²⁺ and calcineurin-derived translocation of, key transcription factor such as NFAT in the expression of cytokines including IFNγ.

In conclusion, our findings suggest that one mechanism by which NK cell education operates is through maintenance of granularity under the influence of continuous inhibitory receptor ligand interactions. Accumulation of effector potential in granular structures allows the cell to separate priming and target cell acquisition, and consequently, the cytolytic machinery can operate independently of transcription at the point of cell-to-cell contact. Furthermore, our data support the proposal that it may be possible to boost NK cell functionality through targeted manipulation of the granular matrix and Ca²⁺ homeostasis within lysosome-related organelles.

### Example 2—Exemplary Formulations and Dosage Regimes of Agents of the Invention

### Mefloquine:

Mefloquine is administered orally in the form of a tablet. The tablet contains mefloquine in the form of mefloquine hydrochloride and poloxamer, microcrystalline cellulose, lactose monohydrate, maize starch, crospovidone, ammonium calcium alginate, talc and magnesium stearate.

Mefloquine is administered to a subject in one of the following dosage regimes:
A single dose of 1250 mg mefloquine.
A first dose of 750 mg mefloquine followed 6-12 hours later by a second dose of 500 mg mefloquine.
A weekly dose of 250 mg mefloquine.

The mefloquine may be administered to a subject suffering from a cancer or an autoimmune disorder.

### Apilimod:

Apilimod is administered orally in the form of a tablet. The tablet contains apilimod in the form of apilimod mesylate. The tablet also contains other standard compounds used in tablet formulation.

Apilimod is administered to a subject in one of the following dosage regimes:
50 mg daily.
100 mg daily.

The apilimod may be administered to a subject suffering from a cancer or an autoimmune disorder.

### Example 3—Ex Vivo Treatment of NK Cells for Enhancement of Effector Functions

NK cells are purified directly from peripheral blood or buffy coats using clinical magnetic separation. Purified NK cells are either used directly or maintained on IL-15 (1-5 ng/mL) for up to 48 hours before infusion.

Alternatively, purified NK cells may be expanded in vitro using protocols that are designed to skew the NK cell population towards a desired reactivity (see for example the teachings of WO 2014/037422).

NK cells to be used for infusion are harvested and incubated in (clinical) medium containing 10 μM vacuolin-1 for 1-4 hours, washed, and administered immediately.

Alternatively, the NK cells are treated with either 0.5 μM Apilimod, 4 μM YM-201636 or 1 μM APY0201 instead of vacuolin-1.

Immune effector cells are contacted with a non-cell-permeable PIKfyve inhibitor in combination with TPCS2a. Photochemical internalisation is then induced using light, and the cells then administered to the subject.

Immune effector cells are treated with U18666/thapsigargin to simulate total degranulation. This effectively resets the cells to allow for adaptation to the new host.

1. A method of preparing a granular immune effector cell for adoptive cell therapy, the method comprising up-regulating the activity of the granular immune effector cell by contacting the cell ex vivo with an agent which modulates the size, content and/or number of secretory lysosomes in the cell, or otherwise modulates the signalling capacity of the secretory lysosomes in the cell.

2. The method of claim 1, wherein the granular immune effector cell is contacted with the agent which modulates the size, content and/or number of secretory lysosomes in the cell, or otherwise modulates the signalling capacity of the secretory lysosomes in the cell, in combination with a ligand or agonist of an inhibitory receptor expressed by the cell and/or an activator of a signalling pathway downstream of the inhibitory receptor.

3. An in vitro or ex vivo method of up-regulating the activity of a granular immune effector cell, comprising contacting the cell with an agent which modulates the size, content and/or number of secretory lysosomes in the cell, or otherwise modulates the signalling capacity of the secretory lysosomes in the cell, in combination with a ligand or agonist of an inhibitory receptor expressed by the cell and/or an activator of a signalling pathway downstream of the inhibitory receptor.

4. The method of any one of claims 1 to 3, wherein the activity is cytotoxic activity and/or cytokine production.

5. The method of any one of claims 1 to 4, wherein the agent prevents lysosomal fission.

6. The method of claim 5, wherein the agent is or comprises vacuolin-1, YM201636, Apilimod and/or APY0201.

7. The method of any one of claims 1 to 4, wherein the agent increases the level of Ca²⁺ in the secretory lysosomes of the cell, or otherwise increases the capacity of the secretory lysosomes to channel or buffer calcium responses.

8. The method of any one of claim 1 to 5 or 7, wherein the agent modulates gene expression.

9. The method of claim 8, wherein:
(i) the agent is an RNA molecule which mediates RNAi or is an agent for use in gene editing, preferably by the CRISPR/Cas9 system; or
(ii) the agent increases or activates expression of one or more genes which encode components of the secretory lysosome matrix or which encode enzymes which mediate synthesis and/or transport of said components.

10. The method of claim 9, wherein the agent modulates the expression of one or more signalling pathways up-stream of the secretory lysosomes in the cell.

11. The method of claim 10, wherein the agent reduces or inactivates expression of one or more genes selected from CD38, CD31, TRPM2, TRPML1, TRPML2, RyR, TPC1, TPC2, and PIKFYVE.

12. The method of claim 9, wherein the agent increases or activates expression of one or more genes selected from SRGN, CHST11, CHST12, NDST2, CST7, GNPTAB, M6PR, CHGA, CHGB or VWF.

13. The method of any one of claims 1 to 12, wherein the agent targets the immune effector cell at the priming stage, during effector development.

14. The method of any one of claims 1 to 13, wherein the agent modulates cell-to-cell interactions.

15. The method of any one of claims 2 to 14, wherein the inhibitory receptor is selected from KIR, PD-1, TIGIT, TIM-3, and NKG2A, preferably wherein the agonist is an antibody.

16. The method of any one of claims 2 to 14, wherein the activator of a signalling pathway downstream of an inhibitory receptor is an agonist of SHP-1, c-Cbl, Cbl-b or c-Abl or an antagonist of Akt, PI3K, Syk, Vav, PLC-g1, PLC-g2 or LAT.

17. The method of any one of claims 1 to 16, wherein the granular immune effector cell is a T-cell or an innate lymphoid cell (ILC).

18. The method of claim 17, wherein the T-cell is a CD4⁺ T-helper cell, a cytotoxic T-cell or a Treg, or wherein the ILC is an NK cell.

19. The method of any one of claims 1 to 18, wherein the granular immune effector cell is derived from an induced pluripotent stem cell.

20. The method of claim 18 or 19, wherein the granular immune effector cell is an NK-cell, and the NK-cell is expanded by stimulating one or both of the paired receptors CD94/NKG2C and CD94/NKG2A, to produce a population of cells.

21. A cell or population of cells produced by the method of any one of claims 1 to 20.

22. The cell or population of cells of claim 21, wherein the cell or population of cells is further modified to modulate its function, preferably wherein the cell or population of cells is modified to express a chimeric antigen receptor or a T-cell receptor, and/or expression of a chemokine receptor on the cell or population of cells has been modified.

23. A pharmaceutical composition comprising a cell or population of cells as defined in claim 21 or 22 together with one or more pharmaceutically acceptable diluents, carriers or excipients.

24. A cell, population of cells or pharmaceutical composition as defined in any one of claims 21 to 23 for use in therapy, preferably adoptive cell therapy.

25. A method of treatment comprising administering a cell, population of cells or pharmaceutical composition as defined in any one of claims 21 to 23 to a subject, preferably wherein said treatment is adoptive cell therapy, preferably wherein said subject is human.

26. The cell, population of cells or pharmaceutical composition for use according to claim 24, or the method of claim 25, wherein the cell is a cytotoxic T-cell, a CD4+ T-helper cell or an NK cell, the population of cells is a population thereof or the pharmaceutical composition comprises such a cell or population of cells, and wherein said therapy or treatment is for cancer, an immunodeficiency or an infection.

27. Use of a cell or a population of cells as defined in claim 22 or 23 in the manufacture of a medicament for use in treating cancer, an immunodeficiency or an infection, wherein the cell is a cytotoxic T-cell, a CD4+ T-helper cell or an NK cell or the population of cells is a population thereof.

28. The cell, population of cells or pharmaceutical composition for use according to claim 26, the method of claim 26 or the use of claim 27, wherein the cancer is melanoma, lung cancer, breast cancer, neuroblastoma, a haematopoietic cancer, including any adult or childhood leukaemia, such as acute myeloid leukaemia, chronic myeloid leukaemia, acute lymphoid leukaemia or chronic lymphoid leukaemia, or any lymphoma, including Hodgkin's lymphoma and non-Hodgkin's lymphomas, including multiple myeloma, and particularly including refractory lymphoid malignancies, glioblastoma, prostate cancer, ovarian cancer, colorectal cancer, renal cell cancer, pancreatic cancer or myelodysplastic syndrome.

29. The cell, population of cells or pharmaceutical composition for use according to claim 24, or the method of claim 25, wherein the cell is a Treg, the population of cells is a population thereof or the pharmaceutical composition comprises such a cell or population of cells, and wherein said therapy or treatment is for an inflammatory condition, preferably wherein the inflammatory condition is an autoimmune disorder, hemophagocytic lymphohistiocytosis (HLH) or familial hemophagocytic lymphohistiocytosis (FHL) or for graft-versus-host-disease following allogeneic stem cell transplantation.

30. Use of a cell or a population of cells as defined in claim 22 or 23 in the manufacture of a medicament for use in treating an inflammatory condition, preferably wherein the inflammatory condition is an autoimmune disorder, hemophagocytic lymphohistiocytosis (HLH) or familial hemophagocytic lymphohistiocytosis (FHL) or graft-versus-host-disease following allogeneic stem cell transplantation, wherein the cell is a Treg or the population of cells is a population thereof.

31. A kit comprising a first agent as defined in any one of claims 1 or 5 to 14 and a second agent selected from (i) a ligand or agonist of an inhibitory receptor expressed by a granular immune effector cell; or (ii) an activator of a signalling pathway downstream of said inhibitory receptor; said ligand, agonist or activator being as defined in any one of claim 2, 15 or 16.

32. The kit of claim 31, wherein the granular immune effector cell is as defined in any one of claims 17 to 19.
